(19) United States
(12) Patent Application Publication
(10) Pub. No.: US 2020/0332370 A1
(43) Pub. Date: Oct. 22, 2020

(54) Methods for Quantification of Nucleosome Modifications and Mutations at Genomic Loci and Clinical Applications Thereof

(71) Applicant: EPICYPHER, INC, Durham, NC (US)

(72) Inventors: Martis William Cowles, Chapel Hill, NC (US); Michael-Christopher Keogh, Cambridge, MA (US)

(21) Appl. No.: 16/960,640

(22) PCT Filed: Jan. 10, 2019
(86) PCT No.: PCT/US2019/013036

§ 371 Date: Jul. 8, 2020

Related U.S. Application Data
(60) Provisional application No. 62/615,770, filed on Jan. 10, 2018.

(51) Int. Cl. C12Q 1/6886; C12N 15/10; C12Q 1/6804; C12N 15/11

(52) U.S. Cl. CPC C12Q 1/6886; C12N 15/1093; C12Q 1/6804; C12Q 2600/154; C12Q 2600/156; C12Q 2600/166; C12N 15/11

(57) ABSTRACT

The invention relates to clinical applications of quantitative chromatin mapping assays, such as chromatin immunoprecipitation assays and assays using tethered enzymes (e.g., chromatin immunocleavage (ChIC) and cleavage under targets & release using nuclease (CUT&RUN®)). The methods may be used to detect and quantitate the presence of epigenetic modifications and mutations on nucleosomes (histones and/or DNA) from biological samples, monitor changes in the status of modifications and mutations, monitor the effectiveness of epigenetic and mutation therapies, select suitable treatments for a disease, determine the prognosis of a subject, identify biomarkers of a disease, and screen for agents that modify epigenetic or mutation status. The invention further relates to kits for use in the methods of the invention.

### STATEMENT OF PRIORITY

This application claims the benefit of U.S. Provisional Application Ser. No. 62/615,770, filed Jan. 10, 2018, the entire contents of which are incorporated by reference herein.

### FIELD OF THE INVENTION

The invention relates to clinical applications of quantitative chromatin mapping assays, such as chromatin immunoprecipitation assays and assays using tethered enzymes (e.g., chromatin immunocleavage (ChIC) and cleavage under targets & release using nuclease (CUT&RUN®)). The methods may be used to detect and quantitate the presence of epigenetic modifications and mutations on nucleosomes (histones and/or DNA) from biological samples, monitor changes in the status of modifications and mutations, monitor the effectiveness of epigenetic and mutation therapies, select suitable treatments for a disease, determine the prognosis of a subject, identify biomarkers of a disease, and screen for agents that modify epigenetic or mutation status. The invention further relates to kits for use in the methods of the invention.

### BACKGROUND OF THE INVENTION

Cooperative function between regulatory proteins, histone post-translational modifications (PTMs), and chromatin structure represents a complex, systems-level signaling network. Numerous chromatin regulators are linked to a diverse collection of human pathologies, including leukemia (Yoo et al., Int. J. Biol. Sci. 8(1):59 (2012)), colorectal cancer (Ashktorab et al., Dig. Dis. Sci. 54(10:2109 (2009); Benard et al., BMC Cancer 14:531 (2014)), Alzheimer's disease (Hendrickx et al., PLoS One 9(6):e99467 (2014)) and Huntington's disease (Moumne et al., Front. Neurol. 4:127 (2013)). As a result, the catalyzed targets of these enzymes (i.e. histone PTMs) are emerging as useful disease indicators (Khan et al., World J. Biol. Chem. 6(4):333 (2015); Chervona et al., Am. J. Cancer Res. 2(5):589 (2012)). To date, there are several FDA-approved epigenetic-targeting drugs on the market for the treatment of cancer, with many more therapeutics targeting chromatin regulation entering preclinical development and Phase I/II clinical trials (Jones et al., Nat. Rev. Genet. 1700:630 (2016)).

The inability to directly detect and quantify patient response remains a formidable technical hurdle that continues to stunt epigenetic drug development. Defining a patient's unique epigenetic background and then monitoring how this landscape is altered in response to therapy could be extremely valuable to select, stratify and assess responses in patients based on their unique genetic and epigenetic features. However, current tools used to quantify histone PTMs are insufficiently reliable (i.e. quantitative, robust, etc.) for clinical studies. ChIP uses antibodies to enrich for nucleosomes that contain specific histone PTMs; the associated DNA is then isolated and mapped to specific genomic loci using qPCR or next-generation sequencing (NGS), respectively, providing a local or genome-wide view of the PTM under study. However, the ChIP-seq approach is semi-quantitative at best (Park et al., Nat. Rev. Genet. 10(10):669 (2009)). Alternatively, ELISA can be used, but this approach is limited to quantifying global PTM changes (i.e. not at selected genomic loci), and thus lacks the resolution and sensitivity to identify/measure cancer-specific biomarkers. Consequently, disease progression and any patient response to epigenetic-targeted therapy are routinely monitored indirectly (e.g. by measuring changes in downstream metabolites, gene expression, etc.). As a result, patient-specific epigenetic backgrounds and any direct quantification of therapeutic responses are currently absent from preclinical/clinical drug development pipelines.

Of note, new chromatin mapping methods have been developed that tether enzymes to genomic regions, resulting in release, enrichment, and subsequent analysis of target material (e.g., the DamID, ChIC, ChEC, and CUT&RUN® approaches). The CUT&RUN (Cleavage Under Target and Release Using Nuclease) method (PCT/US2018/052707) expands upon the previous Chromatin ImmunoCleavage method (ChIC; U.S. Pat. No. 7,790,379) with the development of robust protocols on intact cells using a solid support. CUT&RUN® and ChIC use a factor-specific antibody to tether a fusion protein of protein A and micrococcal nuclease (pA-MN) to genomic binding sites in intact cells, which is then activated by the addition of calcium to cleave DNA. pA-MN provides a cleavage tethering system for antibodies to a PTM, transcription factor, or chromatin protein of interest. CUT&RUN assays produce high quality and reproducible genome-wide PTM mapping data using as few as 100 cells and 3 million reads. Despite these remarkable advances in chromatin mapping technology (vs. traditional ChIP), sample variability and the inability to monitor antibody performance remain formidable technical barriers.

Recently, a new quantative approach was developed for ChIP, termed ICeChIP (Internal Standard Calibrated ChIP (Grzybowski et al., Mol. Cell, 2015. 58(5):886 (2015)) and US Publication No. 2016/0341743. This approach has been commercialized under the names CAP-ChIP® (Calibration and Antibody Profiling) and SNAP-ChIP® (Sample Normalization and Antibody Profiling). This technology utilizes DNA barcoded designer nucleosomes (dNucs) carrying specific histone PTMs as internal control standards for sample normalization and calibration. Barcoded dNucs are spiked into fragmented chromatin samples at various concentrations (the relative amounts encoded in their barcode sequences), then nucleosomes from this pool (cell derived and dNuc) are captured with a bead-immobilized antibody (specific for the PTM of interest). After immunoprecipitation, NGS (or qPCR) data is analyzed for the number of reads detected for: 1) each barcode; and, 2) sample DNA in both the input and IP-captured pools. Read numbers for the IP can then be normalized to input concentration for each barcoded dNuc, providing a standard curve for the direct quantitation of sample DNA reads. Barcoded dNucs serve as direct calibrators because they are subject to the same sources of variability experienced by the sample chromatin during ChIP processing and represent the endogenous antibody target, modified mononucleosomes.

There is a need in the art for reliable and robust methods for quantifying histone PTMs in biological samples for use in clinical applications and drug development.

### SUMMARY OF THE INVENTION

The present invention relates to the clinical application of barcoded recombinant designer nucleosomes as spike-in controls for quantitative chromatin mapping assays (e.g., ChIP, ChIC, or CUT&RUN) that monitor histone PTMs, chromatin associated proteins (e.g., transcription factors, chromatin binding proteins, chromatin remodelers, etc.), and/or mutations in patient samples before and after targeted epigenetic therapy and other treatments. The ability to directly quantify chromatin modifications and regulator proteins genome-wide provides a powerful readout of epigenetic therapeutic effectiveness as well as enables the development of companion diagnostics for disease therapy. As such, this approach will be useful for both drug development and clinical applications.

The chromatin assays useful in the present invention may be any chromatin assay known in the art that produces quantitative results. Examples include, without limitation, the CUT&RUN assay (PCT/US2018/052707), the ChIC assay (U.S. Pat. No. 7,790,379), and the ICeChIP assay (WO 2015/117145). Each of these references are incorporated herein in their entirety.

In some embodiments, the quantitative chromatin assays are chromatin immunoprecipitation assays. Thus, one aspect of the invention relates to a method for detecting and quantitating the presence of an epigenetic modification or a mutation at an epitope of a core histone at a specific genomic locus in chromatin from a biological sample of a subject, the method comprising:

a) isolating a biological sample from the subject;
b) preparing a library of native nucleosomes from chromatin of the biological sample, wherein the library comprises nucleosomes comprising the core histone having the epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library; and
g) determining the density of the epitope of the core histone at the genomic locus by comparing the relative genomic abundance to the standard capture efficiency;

thereby detecting and quantitating the presence of an epigenetic modification or mutation at the epitope.

Another aspect of the invention relates to a method for determining and quantitating the epigenetic or mutation status of a specific genomic locus in chromatin from a biological sample of a subject having a disease or disorder, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library; and
g) determining the density of the epitope of the core histone at the genomic locus by comparing the relative genomic abundance to the standard capture efficiency;

thereby determining and quantitating the epigenetic or mutation status of the genomic locus.

A further aspect of the invention relates to a method for monitoring changes in epigenetic or mutation status over time at a specific genomic locus in chromatin from a biological sample of a subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) repeating steps a) to g) at least one time;

thereby monitoring changes in epigenetic or mutation status over time at the genomic locus.

An additional aspect of the invention relates to a method for monitoring the effectiveness of an epigenetic therapy or mutation therapy in a subject having a disease or disorder associated with epigenetic modifications or mutations, the method comprising monitoring changes in epigenetic or mutation status over time at a specific genomic locus in chromatin from a biological sample of the subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) repeating steps a) to g) at least one time after the onset of the epigenetic therapy or mutation therapy;

thereby monitoring the effectiveness of the epigenetic therapy or mutation therapy in the subject.

Another aspect of the invention relates to a method for selecting a suitable treatment for a subject having a disease or disorder associated with epigenetic modifications or mutations based on the epigenetic or mutation status of a specific genomic locus in chromatin from a biological sample of the subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) selecting a suitable treatment based on the epigenetic or mutation status of the epitope of the core histone.

A further aspect of the invention relates to a method for determining a prognosis for a subject having a disease or disorder associated with epigenetic modifications or mutations based on the epigenetic or mutation status of a specific genomic locus in chromatin from a biological sample of the subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope, and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) determining the prognosis of the subject based on the epigenetic or mutation status of the epitope of the core histone.

An additional aspect of the invention relates to a method for identifying a biomarker of a disease or disorder associated with epigenetic modifications or mutations based on the epigenetic or mutation status of a specific genomic locus in chromatin from a biological sample of the subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) correlating the epigenetic or mutation status of the genomic locus with the disease or disorder associated with epigenetic modifications or mutations; thereby identifying a biomarker of the disease or disorder associated with epigenetic modifications or mutations.

Another aspect of the invention relates to a method of screening for an agent that modifies the epigenetic or mutation status of a specific genomic locus in chromatin from a biological sample of a subject, the method comprising determining the epigenetic or mutation status of the genomic locus in the presence and absence of the agent;

wherein determining the epigenetic or mutation status of the genomic locus comprises:
a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library; and
g) determining the density of the epitope of the core histone at the genomic locus by comparing the relative genomic abundance to the standard capture efficiency;

wherein a change in the epigenetic or mutation status of the genomic locus in the presence and absence of the agent identifies an agent that modifies the epigenetic or mutation status of the genomic locus.

In some embodiments, the quantitative chromatin assays are chromatin mapping assays using tethered enzymes. Thus, one aspect of the invention relates to a method for detecting and quantitating the presence of an epigenetic modification or a mutation at an epitope of a core element at a specific genomic locus in chromatin from a biological sample of a subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA; and
   j) detecting and quantitating the presence of the epitope at the genomic locus by comparing its abundance relative to the nucleosome standard.

Another aspect of the invention relates to a method for determining and quantitating the epigenetic or mutation status of an epitope of a core element at a specific genomic locus in chromatin from a biological sample of a subject having a disease or disorder, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA; and
   j) detecting and quantitating the presence of the epitope at the genomic locus by comparing its abundance relative to the nucleosome standard;
   thereby determining and quantitating the epigenetic or mutation status of the genomic locus.

A further aspect of the invention relates to a method for monitoring changes in epigenetic or mutation status over time of an epitope of a core element at a specific genomic locus in chromatin from a biological sample of a subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard;
   k) repeating steps a) to j) at least one time;
   thereby monitoring changes in epigenetic or mutation status over time at the genomic locus.

An additional aspect of the invention relates to a method for monitoring the effectiveness of an epigenetic therapy or mutation therapy in a subject having a disease or disorder associated with epigenetic modifications or mutations, the method comprising monitoring changes in epigenetic or mutation status over time of an epitope of a core element at a specific genomic locus in chromatin from a biological sample of the subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard;
   k) repeating steps a) to j) at least one time;
   thereby monitoring the effectiveness of the epigenetic therapy or mutation therapy in the subject.

Another aspect of the invention relates to a method for selecting a suitable treatment for a subject having a disease or disorder associated with epigenetic modifications or mutations based on the epigenetic or mutation status of an epitope of a core element at a specific genomic locus in chromatin from a biological sample of the subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard;
   k) selecting a suitable treatment based on the epigenetic or mutation status of the epitope of the core element.

A further aspect of the invention relates to a method for determining a prognosis for a subject having a disease or disorder associated with epigenetic modifications or mutations based on the epigenetic or mutation status of an epitope of a core element at a specific genomic locus in chromatin from a biological sample of the subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard; and
   k) determining the prognosis of the subject based on the epigenetic or mutation status of the epitope of the core element.

An additional aspect of the invention relates to a method for identifying a biomarker of a disease or disorder associated with epigenetic modifications or mutations based on the epigenetic or mutation status of an epitope of a core element at a specific genomic locus in chromatin from a biological sample of the subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard; and
   k) correlating the epigenetic or mutation status of the genomic locus with the disease or disorder associated with epigenetic modifications or mutations; thereby identifying a biomarker of the disease or disorder associated with epigenetic modifications or mutations.

Another aspect of the invention relates to a method of screening for an agent that modifies the epigenetic or mutation status of an epitope of a core element at a specific genomic locus in chromatin from a biological sample of a subject, the method comprising determining the epigenetic or mutation status of the genomic locus in the presence and absence of the agent;
wherein determining the epigenetic or mutation status of the genomic locus comprises:
   a) isolating a biological sample from the subject;
   b) binding a cell, nucleus, organelle, or tissue comprising the core element having the epitope from the biological sample to a solid support;
   c) permeabilizing the cell, nucleus, organelle, or tissue;
   d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA; and
   j) detecting and quantitating the presence of the epitope at the genomic locus by comparing the relative genomic abundance to the nucleosome standard;

wherein a change in the epigenetic or mutation status of the genomic locus in the presence and absence of the agent identifies an agent that modifies the epigenetic or mutation status of the genomic locus.

A further aspect of the invention relates to kits comprising a panel of designer nucleosomes, each nucleosome comprising one or more disease-associated epigenetic modifications or histone mutations.

These and other aspects of the invention are set forth in more detail in the description of the invention below.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of nucleosomes, and transiently and stably transfected cells. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

The term “consisting essentially of” as used herein in connection with a nucleic acid or protein means that the nucleic acid or protein does not contain any element other than the recited element(s) that significantly alters (e.g., more than about 1%, 5% or 10%) the function of interest of the nucleic acid or protein.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-natural amino acid. As used herein, the terms encompass amino acid chains of any length, including full length proteins, wherein the amino acid residues are linked by covalent peptide and/or pseudopeptide bonds.

A “nucleic acid” or “nucleotide sequence” is a sequence of nucleotide bases, and may be RNA, DNA or DNA-RNA hybrid sequences (including both naturally occurring and non-naturally occurring nucleotides), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

By “substantially retain” a property, it is meant that at least about 75%, 85%, 90%, 95%, 97%, 98%, 99% or 100% of the property (e.g., activity or other measurable characteristic) is retained.

The term “epitope” refers to any site on a biomolecule that can evoke binding of an affinity reagent. The affinity reagent might recognize a linear sequence of a biomolecule or biomolecule fragment, the shape of biomolecule or biomolecule fragment, a chemo-physical property of a biomolecule or biomolecule fragment, or a combination of these.

“Amino acids” may be referred to herein by either their commonly known three letter symbols or by the one-letter symbols recommended by the IUPAC-IUB Biochemical Nomenclature Commission. Amino acid residues in proteins or peptides are abbreviated as follows: phenylalanine is Phe or F; leucine is Leu or L; isoleucine is Ile or I; methionine is Met or M; valine is Val or V; serine is Ser or S; proline is Pro or P; threonine is Thr or T; alanine is Ala or A; tyrosine is Tyr or Y; histidine is His or H; glutamine is Gln or Q; asparagine is Asn or N; lysine is Lys or K; aspartic acid is Asp or D; glutamic Acid is Glu or E; cysteine is Cys or C; tryptophan is Trp or W; arginine is Arg or R; and glycine is Gly or G.

The term “amino acid” refers to naturally occurring and non-natural amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refers to compounds that have the same basic chemical structure as a naturally occurring amino acid, i.e., a carbon that is bound to a hydrogen, a carboxyl group, an amino group, and an R group, such as, homoserine, norleucine, methionine sulfoxide, and methionine methyl sulfonium. Such analogs have modified R groups (such as, norleucine) or modified peptide backbones, but retain the same basic chemical structure as a naturally occurring amino acid.

As to amino acid sequences, one of skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are known to those of skill in the art. Such conservatively modified variants are in addition to and do not exclude polymorphic variants, interspecies homologs/orthologs, and alleles of the agents described herein.

An “antigen” as used herein may be any structure which is recognized by an antibody or for which recognizing antibodies can be raised. In certain embodiments, antigens may comprise a single amino acid residue or an amino acid fragment of 2 or more residues. In certain embodiments, antigens may comprise modifications of an amino acid, such as acetylation, methylation (e.g., mono-, di-, tri-), phosphorylation, ubiquitination (e.g., mono-, di-, tri-, poly-), sumoylation, ADP-ribosylation, citrullination, biotinylation, and cis-trans isomerization. In certain embodiments, antigens may comprise nucleotide modifications, such as 5-methylcytosine. In other embodiments, antigens may comprise specific mutations, such as point mutations. In yet other embodiments, antigens may comprise wild-type amino acid sequences or nucleotide sequences.

The term “post-translational modification” refers to any modification of a natural or non-natural amino acid that occurs or would occur to such an amino acid after it has been incorporated into a polypeptide chain in vivo or in vitro. Such modifications include, but are not limited to, acylation (e.g., acetyl-, butyryl-, crotonyl-), methylation (e.g., mono-, di-, tri-), phosphorylation, ubiquitination (e.g., mono-, di-, tri-, poly-), sumoylation, ADP-ribosylation, citrullination, biotinylation, and cis-trans isomerization. Such modifications may be introduced synthetically, e.g., chemically, during polypeptide synthesis or enzymatically after polypeptide synthesis or polypeptide purification.

The term “post-transcriptional modification” refers to any modification of a natural or non-natural nucleotide that occurs or would occur to such a nucleotide after it has been incorporated into a polynucleotide chain in vivo or in vitro. Such modifications include, but are not limited to, 5-methylcyosine, 5-hydroxymethylcytosine, 5,6-dihydrouracil, 7-methylguanosine, xanthosine, and inosine.

The term “immunoprecipitation (IP) enrichment” refers to the internal standard reads from the immunoprecipitated sample divided by the internal standard reads from the input sample.

The term “asymmetric” refers to a nucleosome wherein one histone within a dimer of histones contains a post-translational modification. For example, the trimethyl modification is found on lysine 9 of one histone H3 but absent on the second H3 within a dimer.

The term “symmetric” refers to a nucleosome wherein both histones within a dimer of histones contain a post-translational modification. For example, the trimethyl modification is found on lysine 9 of both histone H3.

The present invention relates to the clinical application of CAP-ChIP and SNAP-ChIP for quantitative monitoring of histone PTMs and mutations in patient samples before and after targeted epigenetic therapy and other treatments. The ability to directly quantify HMD genome-wide provides a powerful readout of epigenetic therapeutic effectiveness as well as enables the development of companion diagnostics for disease therapy. As such, this approach will be useful for both drug development and clinical applications.

Thus, one aspect of the present invention relates to a method for detecting and quantitating the presence of an epigenetic modification or a mutation at an epitope of a core histone at a specific genomic locus in chromatin from a biological sample of a subject, the method comprising:

a) isolating a biological sample from the subject;
b) preparing a library of native nucleosomes from chromatin of the biological sample, wherein the library comprises nucleosomes comprising the core histone having the epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library; and
g) determining the density of the epitope of the core histone at the genomic locus by comparing the relative genomic abundance to the standard capture efficiency;

thereby detecting and quantitating the presence of an epigenetic modification or mutation at the epitope. The steps of the method are not limited to the order in which they are recited. For example, step (c) of adding a standard may be carried out before or after step (b), e.g., the standard may be added to the biological sample before step (b) is carried out or it may be added to the library after step (b) is carried out.

A general description of the assay method is as follows. A semi-synthetic nucleosome ladder of standards with a modified or mutated histone (e.g., H3 carrying N6,N6,N6-trimethylation of lysine 4) in defined concentrations (encoded by each unique DNA barcode) is doped into a library of native nucleosomes isolated from human nuclei and released by in nucleo digestion, e.g., with micrococcal nuclease. A sample of the ladder-doped library is then subjected to immunoprecipitation (IP), DNA purification, and characterization of the DNA, e.g., by next-generation sequencing. Another sample of the ladder-doped library is retained as an input sample and is not subject to immunoprecipitation. Here, immunoprecipitation (IP) or “pull-down” refers to a method or technique for purifying chromatin, nucleosomes, DNA-protein complexes, or proteins including one or more epitopes of interest where the epitope is contacted with an affinity reagent specific to an epitope and separated from other components of the library. The affinity reagent may be any reagent that specifically binds to an epitope and suitable for use in a precipitation assay. The affinity reagent may be an antibody or a fragment or derivative thereof. The affinity reagent may be a non-antibody reagent, such as an aptamer or a protein-protein interaction domain. The term “immunoprecipitation” is used broadly herein to encompass non-antibody affinity reagents.

The immunoprecipitated sample and the input sample are subject to a method with the capability to read out and quantify DNA sequences. Recovered DNA fragments are mapped to the relative genomic position based on a reference genome and the abundance of these fragments is measured for every base pair of the genome for DNA recovered from IP (the sample produced through immunoprecipitation using an affinity reagent) and input (the sample not subject to immunoprecipitation). The same read counting from the sequencing data is performed for the unique nucleotide sequences used to make semi-synthetic nucleosomes. The ratio of abundance of semi-synthetic nucleosomes in IP and input is used to measure IP efficiency and the ratio of abundance of DNA fragments for any genomic loci in IP and input is used to measure relative enrichment. The resulting tag counts for the added semi-synthetic nucleosomes constitute a calibration curve to derive histone modification or mutation density for native nucleosomes genome-wide. The average IP-enrichment ratio for the semi-synthetic nucleosome ladder bearing 100% of the modification is used as a scalar correction for native chromatin bearing the same epitope to compute the amount of modification over a desired genomic interval as a ratio of ratios. Subsequently IP efficiency is applied to relative enrichment to measure histone modification density of the histone post-translational modification or mutation with base pair resolution for the span of the whole genome. In some embodiments, protein epitopes having native-like affinity, specificity and avidity include a protein isoform and/or protein having a post-translational modification. For example, the epitope may be the histone modification to whose density is measured in the assay or an epitope having similar binding characteristics. In one embodiment, the protein part of a DNA-protein complex is a core histone octamer complex containing core histones H2A, H2B, H3, and H4. These sequences are described in Patent Application No: US2013/044537, the contents of which are incorporated by reference herein. In order to reproduce native-like affinity, specificity and avidity of the protein epitope for any of the aforementioned core histones can be represented by any histone variant including those in listed in Table 1(a)-1(f). In one embodiment of the invention, the protein epitope may be a fragment of a histone.

In another aspect of the invention, the protein-DNA complexes comprise a standard polynucleotide comprising but not limited to a positioning sequence and a unique bar code identifier sequence. Inclusion of a protein positioning sequence allows for the creation of a DNA-protein complex through specific native-like interaction with protein. In one embodiment, the protein positioning sequence is a nucleosome positioning sequence. In one embodiment, the positioning sequence comprises a natural or synthetic double-stranded DNA sequence of at least 146 base pairs. In one embodiment, the protein positioning sequence is a “601-Widom” sequence-a synthetic nucleosome binding sequence made through a selection of sequences which exhibited affinity toward a nucleosome. While we have mentioned here a “601-Widom” sequence as a nucleosome positioning sequence the present embodiments encompass the use of other such synthetic and native sequences which exhibit affinity toward nucleosomes. In some embodiments, the standard polynucleotide does not comprise a positioning sequence. As long as the standard polynucleotide is capable of forming a stable protein-DNA association with the histones or histone fragments, it may be used in the methods of the invention.

A unique sequence allows for specific identification of a DNA-protein complex in a library or pool of native DNA-protein complexes, i.e., a barcode. In some embodiments, the unique sequence can be substituted with another means of specific recognition, e.g., a polypeptide, fluorophore, chromophore, RNA sequence, locked nucleic acid sequence, affinity tag etc. In one aspect, the unique sequence can be analyzed by any known nucleotide analysis technique, for example, next-generation sequencing, PCR, qPCR, RT-PCR, ddPCR, hybridization, autoradiography, fluorescent labeling, optical density and the use of intercalating fluorescent probes. A unique sequence and a positioning sequence might be the same sequence and serve a dual function as the recognition molecule. The unique sequence may reside at the 5′-end of the positioning sequence, the 3′ end of the positioning sequence, at both ends of the positioning sequence, and/or internal to the positioning sequence.

In some embodiments, a unique sequence is a duplex DNA sequence with minimal length to maintain a Hamming distance of at least 1 from the genomic sequence of the organism that is being investigated and all other sequences that might be found in the sample. In one embodiment, to guarantee robust discrimination of barcodes in the milieu of native genomic sequences, each barcode is made out of two 11 base pair (bp) sequences absent in human and mice genome (Herold et al., BMC Bioinformatics 9:167 (2008)), where 11 bp sequences are the shortest sequence guaranteeing a Hamming distance of at least 1 for human and mice genomes. In another embodiment, the barcode sequence is a sequence not present in the genome of the cell. In another embodiment, the barcode sequence is a sequence not present in nature. While 11 bp are mentioned here as the shortest possible sequence with a Hamming distance of at least 1 for human and mouse there is an unlimited number of longer sequences with a Hamming distance of at least 1 which can be successfully used to serve as aforementioned unique sequences. Moreover the shortest sequence of unique sequence with a Hamming distance of at least 1 for genomes of other organisms might be shorter than 11 bp and as such, shorter sequences than 11 bp might be successfully used for these organisms. The barcode is a molecule, in one embodiment it is DNA, that can be analyzed by known DNA analysis techniques, including but not limited to next-generation sequencing and PCR. The barcode sequence encodes a concentration and/or identity of a given internal standard nucleosome.

In some embodiments, a unique nucleotide sequence indicates the concentration and identity of a given internal standard. In one aspect of the invention, a unique sequence comprises a length of at least or at most 10, 11, 12, 13, 14, 15, 16, 17, 18, 19, 20, 25, 30, 35, 40, 45, 50, 60, 70, 80, 90 or 100 base pairs in length. In yet another embodiment, the total length of the positioning sequence and unique sequence has a length of at least 100 base pairs. In one aspect, the unique sequence is micrococcal nuclease resistant. In one embodiment of the invention the standard molecule comprising but not limited to a positioning sequence and a unique sequence or barcode comprises, consists essentially of, or consists of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; or SEQ ID NO:15. In one embodiment, the standard molecule comprising but not limited to a positioning sequence and a unique sequence or barcode comprises, consists essentially of, or consists of SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:69; SEQ ID NO:70; SEQ ID NO:71; SEQ ID NO:72; SEQ ID NO:73; SEQ ID NO:74; SEQ ID NO:75; SEQ ID NO:76; SEQ ID NO:77; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:86; SEQ ID NO:87; SEQ ID NO:88; SEQ ID NO:89; SEQ ID NO:90; SEQ ID NO:91; SEQ ID NO:92; SEQ ID NO:93; SEQ ID NO:94; SEQ ID NO:95; SEQ ID NO:96; SEQ ID NO:97; SEQ ID NO:98; SEQ ID NO:99; SEQ ID NO:100; SEQ ID NO:101; SEQ ID NO:102; SEQ ID NO:103; SEQ ID NO:104; SEQ ID NO: 105; SEQ ID NO:106 SEQ ID NO:107; SEQ ID NO:108; SEQ ID NO:109; SEQ ID NO:110; SEQ ID NO:111; SEQ ID NO:112; SEQ ID NO:113; SEQ ID NO:114; or SEQ ID NO:115.

In one embodiment of the method of determining epitope density as described herein, a set of the aforementioned semi-synthetic nucleosomes with the standard polynucleotide is doped into a collection of native nucleosomes. The set may comprise semi-synthetic nucleosomes with the standard polynucleotide harboring more than one epitope but comprising at least one epitope of interest. For example, a set of semi-synthetic nucleosomes may harbor a post-translational modification, e.g., H3K9me3, and a conserved or invariant epitope such as the polypeptide sequence of the histone. Alternatively, a set of semi-synthetic nucleosomes may harbor more than one post-translational modification. In another aspect, the set of standards comprises at least one semi-synthetic, reconstituted, or variant-containing DNA-binding protein with native-like affinity, specificity and avidity of a false positive epitope that is different than the epitope of interest. In one embodiment a set of semi-synthetic or variant containing nucleosomes including at least one nucleosome with native-like affinity, specificity and avidity of a true positive epitope and at least one nucleosome with native-like affinity, specificity and avidity of a false positive epitope.

To purify a population of native or semi-synthetic nucleosomes from a pool of protein-DNA complexes one may use an affinity capture step where an affinity reagent recognizes an invariant fragment of the nucleosome, for example the histone. The affinity agent used in the methods of the invention may be any suitable molecule that recognizes and specifically binds to an epitope of interest. In one aspect the affinity reagent contacting the epitope of interest comprises an antibody or a fragment thereof, a monobody, a scFv, an aptamer, a Fab, or a binding peptide. The method of purifying a population of nucleosomes may apply to semi-synthetic nucleosomes alone, native nucleosomes alone, or a native nucleosomes doped with semi-synthetic nucleosomes.

In one embodiment, to perform the methods of the invention a set of the aforementioned internal standards to which a ChIP read-out can be compared, is doped into a collection of native DNA-protein complexes. Below is described how these standards are used to calculate Standard IP efficiency, which in turn can be used to calculate Protein or Epitope Density (PD), Protein Variant Density (PVD), or Protein Modification Density (PMD), depending whether the investigated epitope is an invariant protein fragment, protein isoform, protein post-translational modification, or polynucleotide post-transcriptional modification. Standards based on semi-synthetic or variant containing nucleosomes with native-like affinity, specificity and avidity improve a chromatin immunoprecipitation by allowing one to perform absolute quantification of Histone Modification Density (HMD) or Histone Variant Density (HVD).

Histone Modification Density is a standardized scale and is defined as the apparent percentage of nucleosomes bearing a specific epitope out of all nucleosomes in a given genomic position. Histone Modification Density is expressed on an analog scale ranging between 0%, meaning absence, and 100% meaning saturating presence of the epitope. For example 90% H3K4me3 Histone Modification Density for nucleosome+1 (the first nucleosome downstream of transcription start site) of GAPDH gene should be interpreted that in the population of all histone H3 molecules composing nucleosome+1 at the GAPDH gene promoter, 90% of them bear post translational modification N6,N6,N6-trimethylation of lysine 4 of histone H3 (H3K4me3) and 10% should be free of H3K4me3. While this example was given for a region of the genome spanning a single nucleosome, which is roughly 147 bp, the same can be applied to any span of the genome ranging from a single base pair to the whole genome.

In order to calculate Protein or Epitope density one needs to know four things: genomic locus size, epitope abundance, general protein abundance, and ImmunoPrecipitation efficiency (“IP efficiency”). Genomic locus size is defined by the user and can range from a single base pair to the whole genome. Epitope abundance is defined as the abundance of the epitope over the span of the genomic locus. Abundance is usually inferred by quantifying the amount of DNA bound to the DNA-protein complex as it is stoichiometric to protein and DNA is easy to quantify with numerous methods, e.g., PCR, RT-PCR, ddPCR, next-generation sequencing, hybridization, autoradiography, fluorescent labeling, optical density, intercalating fluorescent probes, etc. However, abundance may also be measured directly by measuring protein concentration through optical density, fluorescence, autoradiography, mass spectrometry, colorimetric assay, polypeptide total decomposition, etc.

Epitope abundance is measured after an affinity capture step in which a specific affinity reagent recognizes the epitope, after which step epitope-affinity reagent complex is separated from unbound population of DNA-protein complexes. Most often epitope-affinity reagent complex is separated from unbound nucleosomes by immobilizing epitope-affinity reagent complex on the surface and washing away the unbound population of DNA-protein complexes. General protein abundance is defined as the abundance of all proteins of a given kind making DNA-complexes within the span of the given genomic locus. General protein abundance is measured with the same methods as epitope abundance.

To purify a population of nucleosomes from other protein-DNA complexes one can use an affinity capture step where an affinity reagent recognizes an invariant fragment of the nucleosome, for example the histone. However, if a given invariant fragment involved in making the protein-DNA complex is dominant over a considered genomic locus size then the affinity capture step for a general protein population can be skipped under the assumption that the population of other protein-DNA complexes is insignificant. The ratio of epitope abundance and general protein abundance should yield epitope density per protein. However, it is rarely the case that the affinity capture step is 100% efficient and if two or more affinity capture steps are utilized their capture efficiencies will rarely be equal to each other. To solve this problem one needs to know the relative IP efficiency between epitope abundance and general protein abundance measurement.

The “IP efficiency” refers to the relative recovery of the epitope between one or more pull-downs. Knowledge of IP efficiency for the standard allows performing absolute quantification by correcting for differences in recovery between one or more pull-downs. In one embodiment, the aforementioned IP efficiency is measured by using a set of the aforementioned standards that has the same affinity, specificity and avidity as the native epitope and which abundance is easy to measure in a complex mixture. These semi-synthetic standards are doped into a pool of native DNA-protein complexes, a sample of which will be subject to affinity capture. Following this step, the aforementioned measurements of epitope abundance and general protein density is performed for the semi-synthetic standards and the pool of native DNA-protein complexes population with one of the mentioned abundance measurement methods. In one embodiment, the set of standards includes standards that are added at differing concentrations. Here the concentration added is uniquely identified by the barcode.

In one embodiment, epitope abundance can be measured through quantification of DNA bound to DNA-protein complexes for standard DNA-protein complexes and native DNA-protein complexes. In one embodiment, the ratio of epitope of a given standard barcode in the IP versus input material for semi-synthetic nucleosomes is equal to Standard IP Efficiency. Alternatively, this Standard IP efficiency may be computed as a ratio of barcode abundance in the epitope-specific IP versus general protein abundance (for histone H3, for example the barcode counts in the anti-H3 general IP). Once IP efficiency is calculated, one may apply this Standard IP efficiency to IP/input DNA or IP-epitope/IP-general protein ratios for any genomic locus. This is calculated by dividing the genomic IP efficiency-ratio of the epitope abundance in the IP (amount of DNA for a given genomic interval captured in the affinity step) to the amount of DNA covering the same interval present in the input-by the Standard IP efficiency. Alternatively, this may be computed as the ratio of a given genomic DNA fragment in the IP divided by the amount of the same species in the general epitope abundance IP for any genomic locus as described above and then dividing by Standard IP efficiency. The resultant value is a Protein or Epitope Density (PD), also known as a Protein Variant Density (PVD), or Protein Modification Density (PMD).

$$PD ( per \text{/} bp ) = \frac{( \frac{IP}{input} ) \cdot 100 \%}{Standard IP efficiency}$$

Another problem challenging analysis of pull-down experiments is the low precision of prediction stemming from off-target specificity of an affinity reagent used in a pull-down assay. The terms “false positive” and “off-target” are synonymous and refer to an epitope that contacts an affinity reagent promiscuously or non-specifically or an incorrect result. The term “true positive” and “on-target” are synonymous and refers to an epitope of interest or correct result.

The prevalence of false positive epitope signals varies between pull-down to pull-down and depends on the quality of affinity reagent (its intrinsic binding affinity for the desired epitope versus its affinity for other related epitopes), the abundance of on-target versus off-target epitope in the native chromatin, the ratio of capacity of affinity reagent and loading levels of DNA-protein complexes in a pull-down, as well as other conditions under which the pull-down is performed. For different affinity reagents, on- and off-target binding both contribute to the apparent ChIP signal to different degrees, although the extent to which either source contributes within a given experiment with conventional ChIP is unknown. In the absence of knowledge of the abundance of off-target binding, one cannot make a decision whether observed epitope abundance is significant or not, which in turn makes use of pull-down in medical diagnostics and research impractical. The inventors have found a method to quantitate IP efficiency of false positive and true positive epitopes in a pull-down assay in situ, which improves the precision of data interpretation as Positive Predictive Value (PPV) may be readily calculated. PPV allows for an estimation of minimal abundance of epitope at a certain confidence level to be considered a true positive.

Using the aforementioned methods of calculating IP efficiency and Standard IP efficiency, Positive Predictive Value (PPV), also referred to as Precision, may be calculated. Knowledge of PPV streamlines any data analysis as it allows estimation of whether any difference in Protein Density is significant or not, which is not achievable with currently available methods and techniques.

$$Precision = P P V = \frac{\sum α \cdot {η}_{TP}}{\sum α \cdot {η}_{TP} + \sum β \cdot {η}_{FP}}$$

ηTP is IP efficiency of true positive epitope and a is a given weight of true positive epitope, ηFP is IP efficiency of false positive epitope, also known as off-target epitope and β is a weight of false positive epitope. In the absence of prior knowledge of weight distribution α=β=1. Other variants of this equation exist and use of knowledge of false positive and true positive epitope prevalence can be used in other applications.

There are two alternate ways to calibrate ChIP: global histone modification density calibration using an external standard and direct internal standard calibration. Like the relative internal standard approach that was predominantly employed in this work, these two can yield results expressed in “histone modification density” units, which are equal to apparent ratio of probed epitope to all other epitopes available in the given locus.

Global histone modification density calibration relies on a measurement of the total ratio of modification relative to the amount of histone, for example, knowing the percentage of all H3 that is K4 trimethylated. This global histone modification density, derived from either mass spectrometry or quantitative immunoblot measurements can be then redistributed among all IP peaks corrected for input depth in any given locus. The drawback of this method, apart from the sizable error in making the global abundance measurement (for example, MS accuracy plus the ambiguity of perhaps not observing all potential forms of the modification), is that such external measurements by orthogonal methodologies need to be made from the same nucleosomal sample used in the ChIP, and sample handling losses in both techniques are a considerable source of error. In particular, IP-efficiency is never 100% (in practice this can be considerably less), so the degree by which efficiency deviates from the theoretical maximum will be reflected in commensurately inflated values for apparent HMD.

Direct internal standard calibration measures the tag count of a spiked-in barcoded nucleosome standard through the ChIP process, knowing the precise molar concentrations of each internal standard ladder member in the input to extrapolate absolute molar abundance of probed epitope in the original sample. This sort of calibration is limited by the accuracy of counting the number of nuclei subjected to the micrococcal nuclease digest and biased loses that mount on the way from this well quantified number to exhaustively fragmented chromatin isolate. As we recover little more than 80% of the total nucleic acid from digested nuclei under highly optimized digest and isolation conditions, there is some systematic error due to biased genome recovery (Henikoff et al, Nat. Rev. Genet. 9:15 (2009)).

Yet another advantage of this embodiment is the ability to deconvolute the true positive epitope signal from false positive epitope signal, presented here on the example of histone modification density, by solving the following matrix equation: A*x=b. For indicated datasets, CAP-ChIP and SNAP-ChIP-seq tracks were corrected for off-specificity by solving the following matrix equation: A*x=b.

Another embodiment of the invention describes a method to deconvolute the true positive epitope signal from false positive epitope signal, presented here is the example of histone modification density, by solving the following matrix equation: A*x=b

$$A = \vert \begin{matrix}{t}_{a}^{a} & … & {t}_{z}^{a} \\ ⋮ & ⋱ & ⋮ \\ {t}_{a}^{z} & … & {t}_{z}^{z}\end{matrix} \vert , \\ b = \vert \begin{matrix}{HMD}_{1}^{a} & … & {HMD}_{n}^{a} \\ ⋮ & ⋱ & ⋮ \\ {HMD}_{1}^{z} & … & {HMD}_{n}^{z}\end{matrix} \vert , \\ x = \vert \begin{matrix}{HMD ( Cor )}_{1}^{a} & … & H M {D ( Cor )}_{n}^{a} \\ ⋮ & ⋱ & ⋮ \\ {HMD ( Cor )}_{l}^{z} & … & H M {D ( Cor )}_{n}^{z}\end{matrix} \vert$$

where, x is a matrix of corrected HMD scores, A is a matrix of correction factors and b is a matrix of non-corrected HMD scores, where, t is correction factor for specificity toward histone marks from the set of ‘a’ to ‘z’ histone marks (subscript), in the immunoprecipitation using antibody toward a histone mark from the set of ‘a’ to ‘z’ histone marks (superscript); HMD is histone modification density for a given histone mark (‘a’ to ‘z’) from the 1st to the nth locus; HMD(Cor) is corrected histone modification density for a given histone mark from the 1st to the nth locus,

$${t}_{z}^{a} = \frac{\frac{{\sum }_{1}^{N} {IP}_{z}^{a}}{{\sum }_{1}^{N} {input}_{z}}}{\frac{{\sum }_{1}^{N} {IP}_{a}^{a}}{{\sum }_{1}^{N} {input}_{a}}}$$

where, t is a correction factor for specificity toward histone marks from the set of ‘a’ to ‘z’ histone marks (subscript), in the immunoprecipitation using antibody toward a histone mark from the set of ‘a’ to ‘z’ histone marks (superscript); HMD is histone modification density for a given histone mark (‘a’ to ‘z’) from the 1st to the nth locus; HMD(Cor) is corrected histone modification density for a given histone mark from the 1st to the nth locus,

$${t}_{z}^{a} = \frac{\frac{{\sum }_{1}^{N} {IP}_{z}^{a}}{{\sum }_{1}^{N} {input}_{z}}}{\frac{{\sum }_{1}^{N} {IP}_{a}^{a}}{{\sum }_{1}^{N} {input}_{a}}}$$

where, Σ₁^{N} IP and Σ₁^{N} input refer to abundance of the given barcode in the IP or in the input, superscript refers to histone mark toward which antibody was raised, while subscript refers to mark on the semi-synthetic nucleosome that was pulled-down.

The main reasons why conventional ChIP assays have not been adopted in the clinic is that they are often irreproducible due to subtle handling differences and variable antibody specificity, making the % enrichment in the IP widely variant from experiment to experiment, and rendering unbiased comparisons problematic and unreliable. By virtue of having an internal standard that is subject to the steps of ChIP that are sensitive to variation, CAP-ChIP and SNAP-ChIP are far more robust in terms of replication and reliability of results and the numbers are readily compared as HMD is a universal, biologically relevant scale, made by direct in situ comparison to a well-defined internal standard.

Histone modifications and other epigenetic mechanisms are crucial for regulating gene activity and cellular processes. Different histone modifications regulate different processes, such as transcription, DNA replication, and DNA repair. Deregulation of any of these modifications can shift the balance of gene expression leading to aberrant epigenetic patterns and cellular abnormalities. For example, changes in histone post-translational modifications and variants have been detected in various cancers, and aberrant modification patterns are known to be drivers of disease in some cases (Daigle et al., Cancer Cell 20:53 (2011); Chi et al., Nat. Rev. Cancer 10:457 (2010)).

The present invention can be used in the diagnosis, prognosis, classification, prediction of disease risk, detection of recurrence, selection of treatment, and evaluation of treatment efficacy for any disease associated with changes in histone post-translational modifications, post-transcriptional modifications, and mutations, including cancer in a patient, for example, a human patient. Such analyses could also be useful in conjunction with ex vivo culture of patient cells or induced pluripotency stem cells to assess the suitability of a given de-differentiation protocol for producing truly pluripotent stem cells, or the protocols for differentiating stem cells into specific cell types.

In making a diagnosis, prognosis, risk assessment, classification, detection of recurrence or selection of therapy based on the presence, absence, or HMD of a particular histone PTM or mutation, the quantity of the PTM or mutation may be compared to a threshold value that distinguishes between one diagnosis, prognosis, risk assessment, classification, etc., and another. For example, a threshold value can represent the degree of histone methylation that adequately distinguishes between cancer samples and normal biopsy samples with a desired level of sensitivity and specificity. With the use of ICe-ChIP the threshold value will not vary depending on the antibody used or the handling conditions. Threshold value or range can be determined by measuring the particular histone PTM of interest in diseased and normal samples using ICe-ChIP and then determining a value that distinguishes at least a majority of the cancer samples from a majority of non-cancer samples.

The biological sample used in the methods of the invention may be any suitable sample. The biological sample may be, for example, blood, serum, plasma, urine, saliva, semen, prostatic fluid, nipple aspirate fluid, lachrymal fluid, perspiration, feces, cheek swabs, cerebrospinal fluid, cell lysate samples, amniotic fluid, gastrointestinal fluid, biopsy tissue, lymphatic fluid, or cerebrospinal fluid.

In some embodiments, the biological sample comprises cells and the chromatin is isolated from the cells. In certain embodiments, the cells are cells from a tissue or organ affected by a disease or disorder associated with changes in histone post-translational modifications or DNA modifications, e.g., a diseased cell. In some embodiments, the cells are cells from a tissue or organ affected by a disease or disorder associated with mutations in histones, e.g., a diseased cell. The cells may be obtained from the diseased organ or tissue by any means known in the art, including but not limited to biopsy, aspiration, and surgery.

In other embodiments, the cells are not cells from a tissue or organ affected by a disease or disorder associated with changes in histone post-translational modifications or DNA modifications or associated with mutations in histones. The cells may be, e.g., cells that serve as a proxy for the diseased cells. The cells may be cells that are more readily accessible than the diseased cells, e.g., that can be obtained without the need for complicated or painful procedures such as biopsies. Examples of suitable cells include, without limitation, peripheral blood mononuclear cells.

In some embodiments, the biological sample comprises circulating nucleosomes, e.g., nucleosomes that have been released from dying cells. In certain embodiments, the circulating nucleosomes may be from blood cells. In certain embodiments, the circulating nucleosomes may be from cells from a tissue or organ affected by a disease or disorder associated with changes in histone post-translational modifications or DNA modifications or associated with mutations in histones.

The subject may be any subject for which the methods of the present invention are desired. In some embodiments, the subject is a mammal, e.g., a human. In some embodiments, the subject is a laboratory animal, e.g., a mouse, rat, dog, or monkey, e.g., an animal model of a disease. In certain embodiments, the subject may be one that has been diagnosed with or is suspected of having a disease or disorder. In some embodiments, the subject may be one that is at risk for developing a disease or disorder, e.g., due to genetics, family history, exposure to toxins, etc.

In certain embodiments, a plurality of standards is added to the library. In some embodiments, a plurality of standards is added to the library, each standard comprising a reconstituted nucleosome comprising (i) the standard histone or histone fragment having the epitope and (ii) the standard polynucleotide comprising the nucleosome positioning sequence and the barcode identifier sequence, wherein the barcode identifier sequence encodes a concentration parameter indicative of the concentration of the standard added to the library and wherein standards having equivalent concentrations are added to the library. In some embodiments, each PTM or mutation is represented by two or more standards (e.g., 2, 3, 4, 5, 6, 7, 8, 9, or 10), each at the same or similar concentrations. Optionally, duplicate standards each have a different barcode identifier sequence, e.g., for use as an internal standard.

In some embodiments, a plurality of standards is added to the library, each standard comprising a reconstituted nucleosome comprising (i) the standard histone or histone fragment having the epitope and (ii) the standard polynucleotide comprising the nucleosome positioning sequence and the barcode identifier sequence, wherein the barcode identifier sequence encodes a concentration parameter indicative of the concentration of the standard added to the library and wherein standards having at least two differing concentrations are added to the library. In some embodiments, 2, 3, 4, 5, 6, 7, 8, 9, or 10 or more different concentrations of standards are added. Optionally, duplicate standards at each concentration each have a different barcode identifier sequence, e.g., for use as an internal standard.

In certain embodiments, the plurality of standards may further comprise standards comprising reconstituted nucleosomes comprising (i) one or more off-target epitopes and (ii) a standard molecule barcode encoding an off-target epitope identity and concentration parameters indicative to the off-target epitope.

In some embodiments, the method further comprises determining a specificity of off-target capture for the affinity reagent based on one or more capture efficiencies for the off-target epitopes and correcting the density of the epitope of the core histone at the genomic locus based on the specificity of off-target capture.

The epitope may be any epitope on a core histone for which quantitation and/or monitoring is desired. In some embodiments, the epitope is a post-translational modification or a protein isoform. In some embodiments, the epitope of the core histone comprises at least one post-translational amino acid modification, e.g., selected from the group consisting of N-acetylation of serine and alanine; phosphorylation of serine, threonine and tyrosine; N-acylation of lysine (e.g., crotonylation or butyrylation); N6-methylation, N6,N6-dimethylation, N6,N6,N6-trimethylation of lysine; omega-N-methylation, symmetrical-dimethylation, asymmetrical-dimethylation of arginine; citrullination of arginine; ubiquitinylation of lysine; sumoylation of lysine; O-methylation of serine and threonine; phosphorylation of serine, threonine or tyrosine; ADP-ribosylation of arginine, aspartic acid and glutamic acid, and any combination thereof. The modification may be any of those in listed in Table 1(a)-1(f), either singly or in any combination.

In some embodiments, the epitope is a mutation in a core histone, e.g., a mutation associated with a disease or disorder. In some embodiments, the mutation is an oncogenic mutation, e.g., a mutation including, but not limited to, H3K4M, H3K9M, H3K27M, H3G34R, H3G34V, H3G34W, H3K36M, and any combination thereof. The H3 mutants may be based on any variant backbone of H3, e.g., H3.1, H3.2, or H3.3.

In certain embodiments, the methods of the invention may further comprise:

determining an amount of the core histone at the genomic locus in the doped library; and

determining an amount of standard in the doped library.

In some embodiments, the step of determining the amount of the core histone at the genomic locus in the doped library may comprise:

adding a second affinity reagent to the doped library to recover an amount of nucleosomes comprising a second epitope, wherein the second epitope is an invariant epitope present on the core histone, and

determining an amount of polynucleotide in the amount of recovered nucleosomes comprising the second epitope.

In some embodiments, the step of determining the amount of standard in the doped library may comprise:

recovering an amount of reconstituted nucleosome; wherein the reconstituted nucleosome comprises the second epitope, and

determining an amount of the standard molecule in the amount of recovered reconstituted nucleosomes comprising the second epitope.

In these embodiments, the affinity reagent may be an antibody or fragment or variant thereof or a non-antibody reagent directed to the epitope and the second affinity reagent may be an antibody or fragment or variant thereof or a non-antibody reagent directed to the second epitope.

Another aspect of the invention relates to a method for determining and quantitating the epigenetic or mutation status of a specific genomic locus in chromatin from a biological sample of a subject having a disease or disorder, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library; and
g) determining the density of the epitope of the core histone at the genomic locus by comparing the relative genomic abundance to the standard capture efficiency;

thereby determining and quantitating the epigenetic or mutation status of the genomic locus.

The details described above for the method of detecting and quantitating the presence of an epigenetic modification or a mutation apply to this method as well.

A further aspect of the invention relates to a method for monitoring changes in epigenetic or mutation status over time at a specific genomic locus in chromatin from a biological sample of a subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) repeating steps a) to g) at least one time;

thereby monitoring changes in epigenetic or mutation status over time at the genomic locus.

The details described above for the method of detecting and quantitating the presence of an epigenetic modification or a mutation apply to this method as well.

The steps of the method may be repeated as many times as desired to monitor changes in the status of an epigenetic modification or mutation, e.g., 2, 3, 4, 5, 6, 7, 8, 9, 10, 25, 50, or 100 or more times. The method may be repeated on a regular schedule (e.g., daily, weekly, monthly, yearly) or on an as needed basis. The method may be repeated, for example, before, during, and/or after therapeutic treatment of a subject; after diagnosis of a disease or disorder in a subject; as part of determining a diagnosis of a disease or disorder in a subject; after identification of a subject as being at risk for development of a disease or disorder, or any other situation where it is desirable to monitor possible changes in epigenetic modifications or mutations.

An additional aspect of the invention relates to a method for monitoring the effectiveness of an epigenetic therapy or mutation therapy in a subject having a disease or disorder associated with epigenetic modifications or mutations, the method comprising monitoring changes in epigenetic or mutation status over time at a specific genomic locus in chromatin from a biological sample of the subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) repeating steps a) to g) at least one time after the onset of the epigenetic therapy or mutation therapy;

thereby monitoring the effectiveness of the epigenetic therapy or mutation therapy in the subject.

The details described above for the method of detecting and quantitating the presence of an epigenetic modification or a mutation apply to this method as well.

Epigenetic therapies are those designed to alter the epigenetic status of proteins (e.g., histones) or DNA. One example of an epigenetic therapy includes lysine deacetylase inhibitors (formerly termed histone deacetylase inhibitors) (e.g., vorinostat (suberoylanilide hydroxamic acid), CI-994 (tacedinaline), MS-275 (entinostat), BMP-210, M344, NVP-LAQ824, LBH-529 (panobinostat), MGCD0103 (mocetinostat), PXD101 (belinostat), CBHA, PCI-24781, ITF2357, valproic acid, trichostatin A, and sodium butyrate), which are used to treat cutaneous T-cell lymphoma (CTCL) or in clinical trials for the treatment of hematologic and solid tumors, including lung, breast, pancreas, renal, and bladder cancers, melanoma, glioblastoma, leukemias, lymphomas, and multiple myeloma. A further example of an epigenetic therapy is histone acetyltransferase inhibitors (e.g., epigallocatechin-3-gallate, garcinol, anacardic acid, CPTH2, curcumin, MB-3, MG149, C646, and romidepsin). Another example of an epigenetic therapy is DNA methyltransferase inhibitors (e.g., azacytidine, decitabine, zebularine, caffeic acid, chlorogenic acid, epigallocatechin, hydralazine, procainamide, procaine, and RG108), which have been approved for treatment of acute myeloid leukemia, myelodysplastic syndrome, and chronic myelomonocytic leukemia and in clinical trials for treatment of solid tumors. Other epigenetic therapies include, without limitation, lysine methyltransferases (e.g., pinometostat, tazometostat, CPI-1205); lysine demethylases (e.g., ORY1001); arginine methyltransferases (e.g., EPZ020411); arginine deiminases (e.g., GSK484); and isocitrate dehydrogenases (e.g., enasidenib, ivosidenib). See Fischle et al., ACS Chem. Biol. 11:689 (2016); DeWoskin et al., Nature Rev. 12:661 (2013); Campbell et al., J. Clin. Invest. 124:64 (2014); and Brown et al., Future Med. Chem. 7:1901 (2015); each incorporated by reference herein in its entirety.

Mutation therapies include treatments designed to alter the nucleotide sequence of a gene (e.g., encoding a histone). Examples include, without limitation, gene therapy.

The steps of the method may be repeated as many times as desired to monitor effectiveness of the treatment, e.g., 2, 3, 4, 5, 6, 7, 8, 9, 10, 25, 50, or 100 or more times. The method may be repeated on a regular schedule (e.g., daily, weekly, monthly, yearly) or on as needed basis, e.g., until the therapeutic treatment is ended. The method may be repeated, for example, before, during, and/or after therapeutic treatment of a subject, e.g., after each administration of the treatment. In some embodiments, the treatment is continued until the method of the invention shows that the treatment has been effective.

Another aspect of the invention relates to a method for selecting a suitable treatment for a subject having a disease or disorder associated with epigenetic modifications or mutations based on the epigenetic or mutation status of a specific genomic locus in chromatin from a biological sample of the subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) selecting a suitable treatment based on the epigenetic or mutation status of the epitope of the core histone.

The details described above for the method of detecting and quantitating the presence of an epigenetic modification or a mutation apply to this method as well.

The method may be applied, for example, to subjects that have been diagnosed or are suspected of having a disease or disorder associated with epigenetic modifications or mutations. A determination of the epigenetic status or mutation status of an epitope may indicate that the status of an epitope has been modified and an epigenetic therapy or mutation therapy should be administered to the subject to correct the modification. Conversely, a determination that the status of an epitope has not been modified would indicate that an epigenetic therapy or mutation therapy would not be expected to be effective and should be avoided. For example, a determination that a particular genomic locus has been deacetylated may indicate that treatment with a histone deacetylase inhibitor would be appropriate. Similarly, a determination that a particular genomic locus has been hypermethylated may indicate that treatment with a DNA methyltransferase inhibitor would be appropriate.

A further aspect of the invention relates to a method for determining a prognosis for a subject having a disease or disorder associated with epigenetic modifications or mutations based on the epigenetic or mutation status of a specific genomic locus in chromatin from a biological sample of the subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) determining the prognosis of the subject based on the epigenetic or mutation status of the epitope of the core histone.

The details described above for the method of detecting and quantitating the presence of an epigenetic modification or a mutation apply to this method as well.

In some instances, the epigenetic status or mutational status of an epitope is indicative of the prognosis of a disease or disorder associated with epigenetic modifications or mutations. Thus, a determination of the epigenetic status or mutational status of an epitope in a subject that has been diagnosed with or is suspected of having a disease or disorder associated with epigenetic modifications or mutations may be useful to determine the prognosis for the subject. Many such examples are known in the art. One example is prostate cancer and hypermethylation of the glutathione-S transferase P1 (GSTP1) gene promoter, the adenomatous polyposis coli (APC) gene, the genes PITX2, Clorf114, and GABRE˜miR-452˜miR-224, as well as the three-gene marker panel AOX1/Clorf114/HAPLN3 and the 13-gene marker panel GSTP1, GRASP, TMP4, KCNC2, TBX1, ZDHHC1, CAPG, RARRES2, SAC3D1, NKX2-1, FAM107A, SLC13A3, FILIP1L. Another example is prostate cancer and histone PTMS, including, without limitation, increased H3K18Acetylation and H3K4diMethylation associated with a significantly higher risk of prostate tumor recurrence, H4K12Acetylation and H4R3diMethylation correlated with tumor stage, and H3K9diMethylation associated with low-grade prostate cancer patients at risk for tumor recurrence. Another example is the link between overall survival in breast cancer patients and methylation status of CpGs in the genes CREB5, EXPH5, ZNF775, ADCY3, and ADMA8. Another example is glioblastoma and hypermethylation of intronic regions of genes like EGFR, PTEN, NF1, PIK3R1, RB1, PDGFRA, and QKI. A further example is inferior prognosis for colon cancer and methylation status of the promoter of the CNRIP1, FBN1, INA, MAL, SNCA, and SPG20 genes.

Another aspect of the invention relates to a method for identifying a biomarker of a disease or disorder associated with epigenetic modifications or mutations based on the epigenetic or mutation status of a specific genomic locus in chromatin from a biological sample of the subject, the method comprising:

a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative, of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library;
g) determining the density of the epitope of the core histone at the genomic locus by comparing the relative genomic abundance to the standard capture efficiency; and
h) correlating the epigenetic or mutation status of the genomic locus with the disease or disorder associated with epigenetic modifications or mutations; thereby identifying a biomarker of the disease or disorder associated with epigenetic modifications or mutations.

The details described above for the method of detecting and quantitating the presence of an epigenetic modification or a mutation apply to this method as well.

In this method, biological samples of diseased tissue may be taken from a number of patients have a disease or disorder and the epigenetic or mutation status of one or more epitopes determined. Correlations between the epitope status and the occurrence, stage, subtype, prognosis, etc., may then be identified using analytical techniques that are well known in the art.

In any of the methods of the invention, the disease or disorder associated with epigenetic modifications or mutations may be a cancer, a central nervous system (CNS) disorder, an autoimmune disorder, an inflammatory disorder, or an infectious disease.

The cancer may be any benign or malignant abnormal growth of cells, including but not limited to acoustic neuroma, acute granulocytic leukemia, acute lymphocytic leukemia, acute myelogenous leukemia, adenocarcinoma, adrenal carcinoma, adrenal cortex carcinoma, anal cancer, anaplastic astrocytoma, angiosarcoma, basal cell carcinoma, bile duct carcinoma, bladder cancer, brain cancer, breast cancer, bronchogenic carcinoma, cervical carcinoma, cervical hyperplasia, chordoma, choriocarcinoma, chronic granulocytic leukemia, chronic lymphocytic leukemia, chronic myelogenous leukemia, colon cancer, colorectal cancer, craniopharyngioma, cystadenosarcoma, embryonic carcinoma, endometrium cancer, endotheliosarcoma, ependymoma, epithelial carcinoma, esophageal carcinoma, essential thrombocytosis, Ewing's tumor, fibrosarcoma, genitourinary carcinoma, glioblastoma, glioma, gliosarcoma, hairy cell leukemia, head and neck cancer, hemangioblastoma, hepatic carcinoma, Hodgkin's disease, Kaposi's sarcoma, leiomyosarcoma, leukemia, liposarcoma, lung cancer, lymphangioendotheliosarcoma, lymphangiosarcoma, lymphoma, malignant carcinoid carcinoma, malignant hypercalcemia, malignant melanoma, malignant pancreatic insulinoma, mastocytoma, medullar carcinoma, medulloblastoma, melanoma, meningioma, mesothelioma, multiple myeloma, mycosis fungoides, myeloma, myxoma, myxosarcoma, neuroblastoma, non-Hodgkin's lymphoma, non-small cell lung carcinoma, oligodendroglioma, osteogenic sarcoma, ovarian cancer, pancreatic cancer, papillary adenosarcoma, papillary sarcoma, pinealoma, polycythemia vera, primary brain carcinoma, primary macroglobulinemia, prostate cancer, rectal cancer, renal cell carcinoma, retinoblastoma, rhabdomyosarcoma, sebaceous gland sarcoma, seminoma, skin cancer, small cell lung carcinoma, soft-tissue sarcoma, squamous cell carcinoma, stomach carcinoma, sweat gland carcinoma, synovioma, testicular carcinoma, throat cancer, thyroid carcinoma, and Wilms' tumor.

CNS disorders include genetic disorders, neurodegenerative disorders, psychiatric disorders, and tumors. Illustrative diseases of the CNS include, but are not limited to, Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder, mania, manic psychosis,), schizophrenia, schizoaffective disorder, schizophreniform disorder, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions, psychosis not otherwise specified (Psychosis NOS),), dementia, aging, paranoia, attention deficit disorder, psychosexual disorders, sleeping disorders, pain disorders, eating or weight disorders (e.g., obesity, cachexia, anorexia nervosa, and bulemia), ophthalmic disorders involving the retina, posterior tract, and optic nerve (e.g., retinitis pigmentosa, diabetic retinopathy and other retinal degenerative diseases, uveitis, age-related macular degeneration, glaucoma), and cancers and tumors (e.g., pituitary tumors) of the CNS.

Autoimmune and inflammatory diseases and disorders include, without limitation, myocarditis, postmyocardial infarction syndrome, postpericardiotomy syndrome, Subacute bacterial endocarditis, anti-glomerular basement membrane nephritis, interstitial cystitis, lupus nephritis, autoimmune hepatitis, primary biliary cirrhosis, primary sclerosing cholangitis, antisynthetase syndrome, sinusitis, periodontitis, atherosclerosis, dermatitis, allergy, allergic rhinitis, allergic airway inflammation, chronic obstructive pulmonary disease, eosinophilic pneumonia, eosinophilic esophagitis, hypereosinophilic syndrome, graft-versus-host disease, atopic dermatitis, tuberculosis, asthma, chronic peptic ulcer, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease, morphea, pemphigus vulgaris, pityriasis lichenoides et varioliformis acuta, Mucha-Habermann disease, psoriasis, systemic scleroderma, vitiligo, Addison's disease, autoimmune polyendocrine syndrome type 1, autoimmune polyendocrine syndrome type 2, autoimmune polyendocrine syndrome type 3, autoimmune pancreatitis, diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, celiac disease, Crohn's disease, irritable bowel syndrome, diverticulitis, microscopic colitis, ulcerative colitis, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease, palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease, dermatomyositis, fibromyalgia, myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff s encephalitis, chronic inflammatory demyelinating polyneuropathy, Guillain-Barré syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with Streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease, Meniere's disease, Behcet's disease, eosinophilic granulomatosis with polyangiitis, giant cell arteritis, granulomatosis with polyangiitis, IgA vasculitis, Kawasaki's disease, leukocytoclastic vasculitis, lupus vasculitis, rheumatoid vasculitis, microscopic polyangiitis, polyarteritis nodosa, polymyalgia rheumatic, urticarial vasculitis, vasculitis, and primary immune deficiency.

The term “infectious diseases,” as used herein, refers to any disease associated with infection by an infectious agent. Examples of infectious agents include, without limitation, viruses and microorganisms (e.g., bacteria, parasites, protozoans, cryptosporidiums). Viruses include, without limitation, Hepadnaviridae including hepatitis A, B, C, D, E, F, G, etc.; Flaviviridae including human hepatitis C virus (HCV), yellow fever virus and dengue viruses; Retroviridae including human immunodeficiency viruses (HIV) and human T lymphotropic viruses (HTLV1 and HTLV2); Herpesviridae including herpes simplex viruses (HSV-1 and HSV-2), Epstein Barr virus (EBV), cytomegalovirus, varicella-zoster virus (VZV), human herpes virus 6 (HHV-6) human herpes virus 8 (HHV-8), and herpes B virus; Papovaviridae including human papilloma viruses; Rhabdoviridae including rabies virus; Paramyxoviridae including respiratory syncytial virus; Reoviridae including rotaviruses; Bunyaviridae including hantaviruses; Filoviridae including Ebola virus; Adenoviridae; Parvoviridae including parvovirus B-19; Arenaviridae including Lassa virus; Orthomyxoviridae including influenza viruses; Poxviridae including Orf virus, molluscum contageosum virus, smallpox virus and Monkey pox virus; Togaviridae including Venezuelan equine encephalitis virus; Coronaviridae including corona viruses such as the severe acute respiratory syndrome (SARS) virus; and Picornaviridae including polioviruses; rhinoviruses; orbiviruses; picodnaviruses; encephalomyocarditis virus (EMV); Parainfluenza viruses, adenoviruses, Coxsackieviruses, Echoviruses, Rubeola virus, Rubella virus, human papillomaviruses, Canine distemper virus, Canine contagious hepatitis virus, Feline calicivirus, Feline rhinotracheitis virus, TGE virus (swine), Foot and mouth disease virus, simian virus 5, human parainfluenza virus type 2, human metapneuomovirus, enteroviruses, and any other pathogenic virus now known or later identified (see, e.g., Fundamental Virology, Fields et al., Eds., 3^{rd} ed., Lippincott-Raven, New York, 1996, the entire contents of which are incorporated by reference herein for the teachings of pathogenic viruses).

Pathogenic microorganisms include, but are not limited to, Rickettsia, Chlamydia, Chlamydophila, Mycobacteria, Clostridia, Corynebacteria, Mycoplasma, Ureaplasma, Legionella, Shigella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., Helicobacter spp., and any other pathogenic microorganism now known or later identified (see, e.g., Microbiology, Davis et al, Eds., 4^{th} ed., Lippincott, New York, 1990, the entire contents of which are incorporated herein by reference for the teachings of pathogenic microorganisms). Specific examples of microorganisms include, but are not limited to, Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum, Bacillus anthracis, Salmonella typhi, Vibrio cholera, Pasteurella pestis (Yersinia pestis), Pseudomonas aeruginosa, Campylobacter jejuni, Clostridium difficile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, Listeria monocytogenes, Shigella flexneri, Anaplasma phagocytophilum, enterotoxic Escherichia coli, and Schistosoma haematobium.

In some embodiments, the disease or disorder includes, but is not limited to, obesity, diabetes, heart disease, autism, fragile X syndrome, ATR-X syndrome, Angelman syndrome, Prader-Willi syndrome, Beckwith Wiedemann syndrome, Rett syndrome, Rubinstein-Taybi syndrome, Coffin-Lowry syndrome Immunodeficiency-centrometric instability-facial anomalies syndrome, α-thalassaemia, leukemia, Cornelia de Langue syndrome, Kabuki syndrome, progressive systemic sclerosis, and cardiac hypertrophy.

A further aspect of the invention relates to a method of screening for an agent that modifies the epigenetic or mutation status of a specific genomic locus in chromatin from a biological sample of a subject, the method comprising determining the epigenetic or mutation status of the genomic locus in the presence and absence of the agent;

wherein determining the epigenetic or mutation status of the genomic locus comprises:
a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library; and
g) determining the density of the epitope of the core histone at the genomic locus by comparing the relative genomic abundance to the standard capture efficiency;

wherein a change in the epigenetic or mutation status of the genomic locus in the presence and absence of the agent identifies an agent that modifies the epigenetic or mutation status of the genomic locus.

The details described above for the method of detecting and quantitating the presence of an epigenetic modification or a mutation apply to this method as well.

The screening method may be used to identify agents that increase or decrease epigenetic modifications or mutations. In some embodiments, the detected increase or decrease is statistically significant, e.g., at least p<0.05, e.g., p<0.01, 0.005, or 0.001. In other embodiments, the detected increase or decrease is at least about 10%, 20%, 30%, 40%, 50%, 60%, 70%, 80%, 90%, 100% or more.

Any compound of interest can be screened according to the present invention. Suitable test compounds include organic and inorganic molecules. Suitable organic molecules can include but are not limited to small molecules (compounds less than about 1000 Daltons), polypeptides (including enzymes, antibodies, and antibody fragments), carbohydrates, lipids, coenzymes, and nucleic acid molecules (including DNA, RNA, and chimeras and analogs thereof) and nucleotides and nucleotide analogs.

Further, the methods of the invention can be practiced to screen a compound library, e.g., a small molecule library, a combinatorial chemical compound library, a polypeptide library, a cDNA library, a library of antisense nucleic acids, and the like, or an arrayed collection of compounds such as polypeptide and nucleic acid arrays.

Any suitable screening assay format may be used, e.g., high throughput screening.

The method may also be used to characterize agents that have been identified as an agent that modifies the epigenetic or mutation status of a specific genomic locus in chromatin. Characterization, e.g., preclinical characterization, may include, for example, determining effective concentrations, determining effective dosage schedules, and measuring pharmacokinetics and pharmacodynamics.

In some embodiments, the quantitative chromatin assays are chromatin mapping assays using tethered enzymes. Thus, one aspect of the invention relates to a method for detecting and quantitating the presence of an epigenetic modification or a mutation at an epitope of a core element at a specific genomic locus in chromatin from a biological sample of a subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA; and
   j) detecting and quantitating the presence of the epitope at the genomic locus by comparing its abundance relative to the nucleosome standard.

Another aspect of the invention relates to a method for determining and quantitating the epigenetic or mutation status of an epitope of a core element at a specific genomic locus in chromatin from a biological sample of a subject having a disease or disorder, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA; and
   j) detecting and quantitating the presence of the epitope at the genomic locus by comparing its abundance relative to the nucleosome standard;
   thereby determining and quantitating the epigenetic or mutation status of the genomic locus.

A further aspect of the invention relates to a method for monitoring changes in epigenetic or mutation status over time of an epitope of a core element at a specific genomic locus in chromatin from a biological sample of a subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard;
   k) repeating steps a) to j) at least one time;
   thereby monitoring changes in epigenetic or mutation status over time at the genomic locus.

An additional aspect of the invention relates to a method for monitoring the effectiveness of an epigenetic therapy or mutation therapy in a subject having a disease or disorder associated with epigenetic modifications or mutations, the method comprising monitoring changes in epigenetic or mutation status over time of an epitope of a core element at a specific genomic locus in chromatin from a biological sample of the subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard;
   k) repeating steps a) to j) at least one time;
   thereby monitoring the effectiveness of the epigenetic therapy or mutation therapy in the subject.

Another aspect of the invention relates to a method for selecting a suitable treatment for a subject having a disease or disorder associated with epigenetic modifications or mutations based on the epigenetic or mutation status of an epitope of a core element at a specific genomic locus in chromatin from a biological sample of the subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard;
   k) selecting a suitable treatment based on the epigenetic or mutation status of the epitope of the core element.

A further aspect of the invention relates to a method for determining a prognosis for a subject having a disease or disorder associated with epigenetic modifications or mutations based on the epigenetic or mutation status of an epitope of a core element at a specific genomic locus in chromatin from a biological sample of the subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard; and
   k) determining the prognosis of the subject based on the epigenetic or mutation status of the epitope of the core element.

An additional aspect of the invention relates to a method for identifying a biomarker of a disease or disorder associated with epigenetic modifications or mutations based on the epigenetic or mutation status of an epitope of a core element at a specific genomic locus in chromatin from a biological sample of the subject, the method comprising:

a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA;
   j) detecting and quantitating the presence of the epitope at the genomic locus by comparing the relative genomic abundance to the nucleosome standard; and
   k) correlating the epigenetic or mutation status of the genomic locus with the disease or disorder associated with epigenetic modifications or mutations; thereby identifying a biomarker of the disease or disorder associated with epigenetic modifications or mutations.

Another aspect of the invention relates to a method of screening for an agent that modifies the epigenetic or mutation status of an epitope of a core element at a specific genomic locus in chromatin from a biological sample of a subject, the method comprising determining the epigenetic or mutation status of the genomic locus in the presence and absence of the agent;

wherein determining the epigenetic or mutation status of the genomic locus comprises:
a) isolating a biological sample from the subject;
b) binding a cell, nucleus, organelle, or tissue comprising the core element having the epitope from the biological sample to a solid support;
c) permeabilizing the cell, nucleus, organelle, or tissue;
d) binding to a solid support a recombinant nucleosome standard comprising the core element having the epitope, the nucleosome standard comprising:
a. a protein octamer, containing two copies each of histones H2A, H2B, H3, and H4, and optionally, linker histone H1;
b. a DNA molecule, comprising:
   i. a nucleosome positioning sequence,
   ii. a DNA barcode;
   iii. a nuclease or transposase recognition sequence; and
c. a binding member linked to the DNA molecule, wherein the binding member specifically binds to a binding partner;
   e) contacting the permeabilized cell, nucleus, organelle, or tissue of c) and the bound nucleosome standard of d) with an affinity reagent that specifically binds to the epitope;
   f) adding an affinity reagent-binding agent linked to a nuclease or transposase;
   g) allowing the nuclease or transposase to cleave DNA in the cell, nucleus, organelle, or tissue and the nuclease or transposase recognition sequence in the nucleosome standard;
   h) separating cleaved DNA;
   i) identifying the cleaved DNA; and
   j) detecting and quantitating the presence of the epitope at the genomic locus by comparing the relative genomic abundance to the nucleosome standard;

wherein a change in the epigenetic or mutation status of the genomic locus in the presence and absence of the agent identifies an agent that modifies the epigenetic or mutation status of the genomic locus.

For each of these tethered enzyme methods, the description above for chromatin immunoprecipitation assays is applicable.

In some embodiments, the DNA molecule comprises a linker between the nucleosome positioning sequence and the binding member that is about 10 to about 80 nucleotides in length, such as about 15 to about 40 nucleotides or about 15 to about 30 nucleotides, wherein the linker comprises the nuclease or transposase recognition sequence.

As used herein, a “core element” is any protein or nucleic acid covalently or non-covalently bound to or part of a nucleosome, including without limitation histones, nucleic acids, transcription factors, chromatin readers, and chromatin remodelers (e.g., writers, erasers), e.g., histone acetyl transferase, histone deacetylase, SWI/SNF, ISWI.

The nucleosome standards will comprise the same target epitope as the one being detected in the biological sample. The nucleosome standards may comprise one or more than one target epitope. The nucleosome standards may be present in a range of concentrations.

In some embodiments, the nuclease or transposase recognition sequence is recognized by an endodeoxyribonuclease, such as micrococcal nuclease, S1 nuclease, mung bean nuclease, pancreatic DNase I, yeast HO endonuclease, a restriction endonuclease, or a homing endonuclease. In some embodiments, the recognition sequence may be a specific sequence that is bound by the nuclease or transposase. In some embodiments, the recognition sequence may be a sequence that is not recognized by the nuclease or transposase based on a specific sequence but has characteristics that cause the sequence to preferably be bound by the nuclease or transposase. In one embodiment, the recognition sequence is an A/T-rich region.

In some embodiments, the nuclease or transposase recognition sequence is recognized by a transposase, such as Tn5, Mu, IS5, IS91, Tn552, Ty1, Tn7, Tn/O, Mariner, P Element, Tn3, Tn1O, or Tn903.

In some embodiments, the binding member and its binding partner are pairings such as biotin with avidin or streptavidin, a nano-tag with streptavidin, glutathione with glutathione transferase, an antigen/epitope with an antibody, polyhistidine with nickel, a polynucleotide with a complementary polynucleotide, an aptamer with its specific target molecule, or Si-tag and silica.

In some embodiments, the binding member is linked to the 5′ and/or 3′ end of the DNA molecule.

In some embodiments, the DNA barcode has a length of about 6 to about 50 basepairs, such as about 7 to about 30 basepairs or about 8 to about 20 basepairs.

In some embodiments, each histone in the nucleosome is independently fully synthetic, semi-synthetic, or recombinant.

In some embodiments, the histone post-translational modifications, mutations, and/or histone variants and/or DNA post-transcriptional modifications are selected from post-translational modification including but not limited to N-acetylation of serine and alanine; phosphorylation of serine, threonine and tyrosine; N-crotonylation, N-acylation of lysine; N6-methylation, N6,N6-dimethylation, N6,N6,N6-trimethylation of lysine; omega-N-methylation, symmetrical-dimethylation, asymmetrical-dimethylation of arginine; citrullination of arginine; ubiquitinylation of lysine; sumoylation of lysine; O-methylation of serine and threonine, ADP-ribosylation of arginine, aspartic acid and glutamic acid; oncogenic mutations (e.g. H3K4M, H3K9M, H3K27M, H3G34R, H3G34V, H3G34W, or H3K36M); post-transcriptional modification including but not limited to 5-methylcytosine, 5-hydroxymethylcytosine, 5-formylcytosine, 5-carboxylcytosine, and 3-methylcytosine; and histone variants (e.g., H3.3, H2A.Bbd, H2A.Z.1, H2A.Z.2, H2A.X, mH2A1.1, mH2A1.2, mH2A2, and TH2B).

In some embodiments, the nucleosome may be part of a panel, wherein the panel comprises at least two nucleosomes comprising different histone post-translational modifications, mutations, and/or histone variants and/or DNA post-transcriptional modifications. In certain embodiments, each nucleosome in the panel comprises a different histone post-translational modification, mutation, and/or histone variant and/or DNA post-transcriptional modification is present at the same concentration in the panel. In certain embodiments, each nucleosome in the panel comprises a different histone post-translational modification, mutation, and/or histone variant and/or DNA post-transcriptional modification is present at multiple concentrations in the panel and the DNA barcode of each nucleosome indicates that concentration at which the nucleosome is present in the panel. In some embodiments, the panel further comprises a synthetic nucleosome which does not comprise a post-translational modification, mutation, or histone variant and/or DNA post-transcriptional modification.

In some embodiments, the nucleosome is part of a polynucleosome, e.g., comprising 2-10 nucleosomes. In certain embodiments, the polynucleosome is part of an array. In some embodiments, the array is part of a pool of arrays, wherein each array comprises a unique histone post-translational modification, mutation, or histone variant and/or DNA post-transcriptional modification.

In some embodiments, the nuclease or transposase of step (f) is inactive and step (g) comprises activating the nuclease or transposase, e.g., by adding an activating ion such as calcium.

In some embodiments, identifying the cleaved DNA comprises subjecting the cleaved DNA to amplification and/or sequencing, such as qPCR, Next Generation Sequencing, or Nanostring.

In some embodiments, the methods further comprise determining the identity of the nucleosome, panel, polynucleosome, array, or pool based on the sequence of the DNA barcode in the cleaved DNA.

In the above methods, the solid support may be, for example, a bead (e.g., a magnetic bead) or a well.

Another aspect of the invention provides reagents and kits including reagents for carrying out one of the methods described herein. The reagents may be included in suitable packages or containers. The kit may include one or more reagents containing standards as described herein for the absolute quantification of true positive and false positive epitopes, for example in a pull-down assay, chromatin immunoprecipitation assay, or chromatin tethered enzyme assay. The kit may also include at least one affinity reagent as described herein, for example an antibody or a fragment or variant thereof. The kit may also include reagents (e.g., primers, probes) for sequencing the barcode identifier sequences. The standards may have native-like affinity, specificity and avidity for a true positive epitope. The kit can also comprise at least one standard with native-like affinity, specificity and avidity of epitope for a false positive epitope.

In some embodiments, the standards include DNA-protein complexes comprising semi-synthetic nucleosomes, made with histones, histone isoforms, histone post-translational modifications, or histone mutations with native-like affinity, specificity and avidity and a barcode identifier sequence. In various embodiments, any variant of core histone sequences, which are known in the art, or post-translational modification, including those defined in Tables 1(a)-1(f), can be installed on the histones that comprise the histone octamer under presumption that native-like affinity, specificity and avidity of epitope is maintained. In one embodiment, a set of standards is comprised of at least a single standard of DNA-complexes with native-like affinity, specificity and avidity of epitope for true positive epitope and multiple standard DNA-complexes with native-like affinity, specificity and avidity of epitope covering a range of possible off-target epitopes (false positive epitopes) present in the native pool of DNA-protein complexes.

In other embodiments, the kit may include one or more wash buffers, (for example, phosphate buffered saline) and/or other buffers in packages or containers. In yet other embodiments, the kits may include reagents necessary for the separation of the captured agents, for example a solid-phase capture reagent including, for example, paramagnetic particles linked to a second antibody or protein-A. The kit may also include reagents necessary for the measurement of the amount of captured standard or sample.

When a kit is supplied, the different components may be packaged in separate containers and admixed immediately before use. Such packaging of the components separately may permit long-term storage without losing the active components' functions. Kits may also be supplied with instructional materials. Instructions may be printed on paper or other substrate, and/or may be supplied as an electronic-readable medium.

In some embodiments, the kit may comprise a panel of standards that represent some or all of the different possibilities of a particular class of PTM, e.g., lysine methylation, lysine acylation, or arginine methylation, e.g., of a single histone or multiple histones. The panel may include some or all of the modifications considered to be relevant to one or more diseases. In some embodiments, the kit may comprise a set of standards that represent most or all of the different possibilities of histone mutations, e.g., oncogenic histone mutations, e.g., of a single histone or multiple histones. The panels may be used to assess the specificity of affinity reagents, monitor technical variability, and normalize experiments. Quantitating the recovery of the standards may also be used as a stop/go decision point for continuing on to the remainder of the assay (e.g., next-generation sequencing).

In some embodiments, each species in the panel may be included multiple times. In some embodiments, each species may be represented more than one time at the same concentration, each iteration of the species having a distinct barcode identifier sequences as a form of internal control. In some embodiments, each species may be represented more than one time at different concentrations, each iteration having a unique barcode identifier sequence that represents the concentration of the standard. Such a concentration series may be used to provide a standard curve for the assay. Each of the concentrations may be represented more than one time, each iteration of the species having a distinct barcode identifier sequences as a form of internal control.

One example of a lysine methylation panel of standards includes some or all of the PTMs selected from H3K4, H3K9, H3K27, H3K36, and H4K20, each potentially represented in the panel having 0, 1, 2, or 3 methyl groups. In one embodiment, the panel may have 16 species (each of the 5 lysine residues each having 1, 2, or 3 methyl groups plus an unmodified standard). In some embodiments, the panel may include duplicates of each standard having distinct barcode identifier sequences as a form of internal control. Thus, the panel may include up to 32 different species. In some embodiments, each of the up to 16 different standards may be represented multiple times at the same or different concentrations with each standard having a unique barcode identifier sequence that represents the concentration of the standard. For example, each standard may be present in the panel in 2, 3, 4, 5, 6, 7, 8, 9, 10 or more different concentrations, each concentration having a different barcode identifier sequence. Thus, a panel may have unique standards in multiples of 8 or 16, e.g., 16, 24, 32, 40, 48, 56, 64, 72, 80, 96, 104, 112, 120, 128, 136, 144, 152, or 160 total species.

One example of an arginine methylation panel of standards includes some or all of the PTMs selected from H2AR2me1, H2AR2me2a, H2AR2me2s, H3R2me1, H3R2me2a, H3R2me2s, H3R8me1, H3R8me2a, H3R8me2s, H3R17me1, H3R17me2a, H4R3me1, H4R3me2a, and H4R3me2s, wherein a is asymmetric and s is symmetric. In one embodiment, the panel may have 15 species (each of the 14 PTMs plus an unmodified standard). In some embodiments, the panel may include duplicates of each standard having distinct barcode identifier sequences as a form of internal control. Thus, the panel may include up to 30 different species. In some embodiments, each of the up to 15 different standards may be represented multiple times at the same or different concentrations with each standard having a unique barcode identifier sequence that represents the concentration of the standard. For example, each standard may be present in the panel in 2, 3, 4, 5, 6, 7, 8, 9, 10 or more different concentrations, each concentration having a different barcode identifier sequence. Thus, a panel may have unique standards in multiples of 15, e.g., 30, 45, 60, 75, 90, 105, 120, 135, or 150 total species.

One example of an lysine acylation panel of standards includes some or all of the PTMs selected from H2AtetraAc, H3K4ac, H3K9ac, H3K9bu, H3K9cr, H3K14ac, H3K18ac, H3K18bu, H3K18cr, H3tetraAc (K4-9-14-18ac), H3K23ac, H3K27ac, H3K27bu, H3K27cr, H3K36ac, H3K56ac, H4K5ac, H4K8ac, H4K12ac, H4K16ac, H4tetraAc (K5-8-12-16ac), and H4K20ac. In one embodiment, the panel may have 23 species (each of the 22 PTMs plus an unmodified standard). In some embodiments, the panel may include duplicates of each standard having distinct barcode identifier sequences as a form of internal control. Thus, the panel may include up to 46 different species. In some embodiments, each of the up to 23 different standards may be represented multiple times at the same or different concentrations with each standard having a unique barcode identifier sequence that represents the concentration of the standard. For example, each standard may be present in the panel in 2, 3, 4, 5, 6, 7, 8, 9, 10 or more different concentrations, each concentration having a different barcode identifier sequence. Thus, a panel may have unique standards in multiples of 23, e.g., 46, 69, 92, 115, 138, 161, 184, 207, or 230 total species.

One example of an oncogenic mutation panel of standards includes some or all of the mutations including, but not limited to, H3K4M, H3K9M, H3K27M, H3G34R, H3G34V, H3G34W, H3K36M, and any combination thereof. The panel may also include wild-type 113. The H3 mutants may be based on any variant backbone of H3, e.g., H3.1, H3.2, or 113.3. Thus, the panel may include up to 8 different species, each with a unique barcode identifier sequence. In some embodiments, the panel may include duplicates of each standard having distinct barcode identifier sequences as a form of internal control. Thus, the panel may include up to 16 different species. In some embodiments, each of the up to 8 different standards may be represented multiple times at the same or different concentrations with each standard having a unique barcode identifier sequence that represents the concentration of the standard. For example, each standard may be present in the panel in 2, 3, 4, 5, 6, 7, 8, 9, 10 or more different concentrations, each concentration having a different barcode identifier sequence. Thus, a panel may have unique standards in multiples of 8 or 16, e.g., 16, 24, 32, 40, 48, 56, 64, 72, 80, 96, 104, 112, 120, 128, 136, 144, 152, or 160 total species.

In some embodiments, the kit is suitable for chromatin assay using tethered enzymes. In some embodiments, the kit comprises the nucleosome, panel, polynucleosome, array, pool or bead of the invention. In some embodiments, the kit further comprises an antibody, aptamer, or other affinity reagent that specifically binds to a histone post-translational modification, mutation, or histone variant or DNA post-transcriptional modification. In some embodiments, the kit further comprises a nuclease or transposase linked to an antibody-binding protein, such as protein A, protein G, a fusion between protein A and protein G, protein L, or protein Y or the like, or to an entity (e.g., a protein) that binds the recognition agent. In some embedment's, the kit further comprises a bead comprising a binding partner to the binding member, such as a magnetic bead.

The foregoing is illustrative of the present invention, and is not to be construed as limiting thereof. The invention is defined by the following claims, with equivalents of the claims to be included therein.

TABLE 1(a)
Post translational modifications for Human Histones H2A
type 1/2/3, H2A.X, H2A.Z and H2A.V Isoform 1/2/3/4/5

| | |
|---|---|
| Position | Description of Modification Type |

Human Histone H2a type 1/2/3

| | |
|---|---|
| 1 | N-acetylserine |
| 1 | Phosphoserine |
| 3 | Citrulline |
| 5 | N6-acetyllysine |
| 36 | N6-crotonyl-L-lysine |
| 118 | N6-crotonyl-L-lysine |
| 119 | N6-crotonyl-L-lysine |
| 120 | Phosphothreonine |
| 126 | N6-crotonyl-L-lysine |
| 13 | Glycyl lysine isopeptide (Lys-Gly) |
| | (interchain with G-Cter in ubiquitin) |
| 15 | Glycyl lysine isopeptide (Lys-Gly) |
| | (interchain with G-Cter in ubiquitin) |
| 119 | Glycyl lysine isopeptide (Lys-Gly) |
| | (interchain with G-Cter in ubiquitin) |

Human Histone H2A.X

| | |
|---|---|
| 1 | N-acetylserine |
| 1 | Phosphoserine |
| 36 | N6-acetyllysine |
| 119 | Phosphoserine |
| 142 | Phosphotyrosine |
| 13 | Glycyl lysine isopeptide (Lys-Gly) |
| | (interchain with G-Cter in ubiquitin) |
| 15 | Glycyl lysine isopeptide (Lys-Gly) |
| | (interchain with G-Cter in ubiquitin) |
| 119 | Glycyl lysine isopeptide (Lys-Gly) |
| | (interchain with G-Cter in ubiquitin) |

Human Histone H2A.Z

| | |
|---|---|
| 1 | N-acetylalanine |
| 4 | N6-acetyllysine |
| 7 | N6-acetyllysine |
| 11 | N6-acetyllysine |
| 13 | N6-acetyllysine |
| 121 | Glycyl lysine isopeptide (Lys-Gly) |
| | (interchain with G-Cter in ubiquitin) |

Human Histone H2A.V Isoform 1/2/3/4/5

| | |
|---|---|
| 4 | N6-acetyllysine |
| 7 | N6-acetyllysine |
| 11 | N6-acetyllysine |
| 121 | Glycyl lysine isopeptide (Lys-Gly) |
| | (interchain with G-Cter in ubiquitin) |

TABLE 1(b)
Post translational modifications for
Human Histone H2A.J and H2B type 1

| | |
|---|---|
| Position | Description of Modification Type |

Human Histone H2A.J

| | |
|---|---|
| 1 | N-acetylserine |
| 1 | Phosphoserine |
| 5 | N6-acetyllysine |
| 120 | Phosphothreonine |
| 122 | Phosphoserine |
| 13 | Glycyl lysine isopeptide (Lys-Gly) |
| | (interchain with G-Cter in ubiquitin) |
| 15 | Glycyl lysine isopeptide (Lys-Gly) |
| | (interchain with G-Cter in ubiquitin) |
| 119 | Glycyl lysine isopeptide (Lys-Gly) |
| | (interchain with G-Cter in ubiquitin) |

Human Histone H2B type 1

| | |
|---|---|
| 1 | N-acetylproline |
| 6 | N6-acetyllysine |
| 6 | N6-crotonyl-L-lysine |
| 12 | N6-acetyllysine |
| 12 | N6-crotonyl-L-lysine |
| 13 | N6-acetyllysine |
| 13 | N6-crotonyl-L-lysine |
| 16 | N6-acetyllysine |
| 16 | N6-crotonyl-L-lysine |
| 17 | N6-acetyllysine |
| 17 | N6-crotonyl-L-lysine |
| 21 | N6-acetyllysine |
| 21 | N6-crotonyl-L-lysine |
| 24 | N6-acetyllysine |
| 24 | N6-crotonyl-L-lysine |
| 35 | N6-crotonyl-L-lysine |
| 37 | Phosphoserine |
| 47 | N6-methyllysine |
| 58 | N6,N6-dimethyllysine |
| 80 | Dimethylated arginine |
| 85 | Phosphoserine |
| 86 | N6,N6,N6-trimethyllysine |
| 86 | N6-acetyllysine |
| 87 | Omega-N-methylarginine |
| 93 | Omega-N-methylarginine |
| 109 | N6-methyllysine |
| 116 | Phosphothreonine |
| 117 | N6-methylated lysine |
| 35 | Glycyl lysine isopeptide (Lys-Gly) |
| | (interchain with G-Cter in ubiquitin) |
| 121 | Glycyl lysine isopeptide (Lys-Gly) |
| | (interchain with G-Cter in ubiquitin) |

TABLE 1(c)
Post translational modifications for
Human Histone H2B type 2/3/F-S

| Position | Description of Modification Type |
|---|---|
| 1 | N-acetylproline |
| 5 | N6-acetyllysine |
| 5 | N6-crotonyl-L-lysine |
| 11 | N6-acetyllysine |
| 11 | N6-crotonyl-L-lysine |
| 12 | N6-acetyllysine |
| 12 | N6-crotonyl-L-lysine |
| 14 | Phosphoserine |
| 15 | N6-acetyllysine |
| 15 | N6-crotonyl-L-lysine |
| 16 | N6-acetyllysine |
| 16 | N6-crotonyl-L-lysine |
| 20 | N6-acetyllysine |
| 20 | N6-crotonyl-L-lysine |
| 23 | N6-acetyllysine |
| 23 | N6-crotonyl-L-lysine |
| 34 | N6-crotonyl-L-lysine |
| 36 | Phosphoserine |
| 46 | N6-methyllysine |
| 57 | N6,N6-dimethyllysine |
| 79 | Dimethylated arginine |
| 85 | N6,N6,N6-trimethyllysine |
| 85 | N6-acetyllysine |
| 86 | Omega-N-methylarginine |
| 92 | Omega-N-methylarginine |
| 108 | N6-methyllysine |
| 115 | Phosphothreonine |
| 116 | N6-methylated lysine |
| 112 | O-linked (GlcNAc) |
| 34 | Glycyl lysine isopeptide (Lys-Gly) |
| | (interchain with G-Cter in ubiquitin) |
| 121 | Glycyl lysine isopeptide (Lys-Gly) |
| | (interchain with G-Cter in ubiquitin) |

TABLE 1(d)
Post translational modifications for Human
Putative Histone H2B type 2-D/2-C

| Position | Description of Modification Type |
|---|---|
| 1 | N-acetylproline |
| 5 | N6-acetyllysine |
| 5 | N6-crotonyl-L-lysine |
| 11 | N6-acetyllysine |
| 11 | N6-crotonyl-L-lysine |
| 12 | N6-acetyllysine |
| 12 | N6-crotonyl-L-lysine |
| 14 | Phosphoserine |
| 15 | N6-acetyllysine |
| 15 | N6-crotonyl-L-lysine |
| 16 | N6-acetyllysine |
| 16 | N6-crotonyl-L-lysine |
| 20 | N6-acetyllysine |
| 20 | N6-crotonyl-L-lysine |
| 23 | N6-acetyllysine |
| 23 | N6-crotonyl-L-lysine |
| 34 | N6-crotonyl-L-lysine |
| 36 | Phosphoserine |
| 46 | N6-methyllysine |
| 57 | N6,N6-dimethyllysine |
| 79 | Dimethylated arginine |
| 85 | N6,N6,N6-trimethyllysine |
| 85 | N6-acetyllysine |
| 86 | Omega-N-methylarginine |
| 92 | Omega-N-methylarginine |

TABLE 1(e)
Post translational modifications for Human
Histone H3.1/H3.1t/H3.2/H3.3/H3.3C

| Position | Modification Type |
|---|---|
| 2 | Asymmetric dimethylarginine |
| 3 | Phosphothreonine |
| 4 | Allysine |
| 4 | N6,N6,N6-trimethyllysine |
| 4 | N6,N6-dimethyllysine |
| 4 | N6-acetyllysine |
| 4 | N6-crotonyl-L-lysine |
| 4 | N6-methyllysine |
| 6 | Phosphothreonine |
| 8 | Citrulline |
| 8 | Symmetric dimethylarginine |
| 9 | N6,N6,N6-trimethyllysine |
| 9 | N6,N6-dimethyllysine |
| 9 | N6-acetyllysine |
| 9 | N6-crotonyl-L-lysine |
| 9 | N6-methyllysine |
| 10 | Phosphoserine |
| 11 | Phosphothreonine |
| 14 | N6-acetyllysine |
| 17 | Asymmetric dimethylarginine |
| 17 | Citrulline |
| 18 | N6-acetyllysine |
| 18 | N6-crotonyl-L-lysine |
| 18 | N6-methyllysine |
| 23 | N6-acetyllysine |
| 23 | N6-crotonyl-L-lysine |
| 23 | N6-methyllysine |
| 27 | N6,N6,N6-trimethyllysine |
| 27 | N6,N6-dimethyllysine |
| 27 | N6-acetyllysine |
| 27 | N6-crotonyl-L-lysine |
| 27 | N6-methyllysine |
| 28 | Phosphoserine |
| 36 | N6,N6,N6-trimethyllysine |
| 36 | N6,N6-dimethyllysine |
| 36 | N6-acetyllysine |
| 36 | N6-methyllysine |
| 37 | N6-methyllysine |
| 41 | Phosphotyrosine |
| 56 | N6,N6,N6-trimethyllysine |
| 56 | N6-acetyllysine |
| 56 | N6-crotonyl-L-lysine |
| 56 | N6-methyllysine |
| 57 | Phosphoserine |
| 64 | N6-methyllysine |
| 79 | N6,N6,N6-trimethyllysine |
| 79 | N6,N6-dimethyllysine |
| 79 | N6-acetyllysine |
| 79 | N6-methyllysine |
| 80 | Phosphothreonine |
| 107 | Phosphothreonine |
| 115 | N6-acetyllysine |
| 122 | N6-acetyllysine |
| 122 | N6-methyllysine |

TABLE 1(f)
Post translational modifications for Human Histone
H3-like centromeric protein A and Human Histone H4

| | |
|---|---|
| Position | Description of Modification Type |

Human Histone H3-like centromeric protein A

| | |
|---|---|
| 6 | Phosphoserine; by AURKA and AURKB |
| 16 | Phosphoserine |
| 18 | Phosphoserine |
| 26 | Phosphoserine |

Human Histone H4

| | |
|---|---|
| 1 | N-acetylserine |
| 1 | Phosphoserine |
| 3 | Asymmetric dimethylarginine |
| 3 | Citrulline |
| 3 | Omega-N-methylarginine |
| 3 | Symmetric dimethylarginine |
| 5 | N6-acetyllysine |
| 5 | N6-crotonyl-L-lysine |
| 8 | N6-acetyllysine |
| 8 | N6-crotonyl-L-lysine |
| 12 | N6-acetyllysine |
| 12 | N6-crotonyl-L-lysine |
| 16 | N6-acetyllysine |
| 16 | N6-crotonyl-L-lysine |
| 20 | N6,N6,N6-trimethyllysine |
| 20 | N6,N6-dimethyllysine |
| 20 | N6-methyllysine |
| 31 | N6-acetyllysine |
| 47 | Phosphoserine |
| 51 | Phosphotyrosine |
| 88 | Phosphotyrosine |
| 91 | N6-acetyllysine |
| 91 | Glycyl lysine isopeptide (Lys-Gly) |
| | (interchain with G-Cter in ubiquitin) |

1. A method for detecting and quantitating the presence of an epigenetic modification or a mutation at an epitope of a core histone at a specific genomic locus in chromatin from a biological sample of a subject, the method comprising:
a) isolating a biological sample from the subject;
b) preparing a library of native nucleosomes from chromatin of the biological sample, wherein the library comprises nucleosomes comprising the core histone having the epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library; and
g) determining the density of the epitope of the core histone at the genomic locus by comparing the relative genomic abundance to the standard capture efficiency;
thereby detecting and quantitating the presence of an epigenetic modification or mutation at the epitope.

2. A method for determining and quantitating the epigenetic or mutation status of a specific genomic locus in chromatin from a biological sample of a subject having a disease or disorder, the method comprising:
a) isolating a biological sample from the subject;
b) preparing a library of native nucleosomes from chromatin of the biological sample, wherein the library comprises nucleosomes comprising a core histone having an epitope and a polynucleotide comprising a nucleotide sequence indicative of the genomic locus;
c) adding a standard to the library to create a doped library; wherein the standard comprises a reconstituted nucleosome comprising (i) a standard histone or histone fragment having the epitope and (ii) a standard polynucleotide comprising a nucleosome positioning sequence and a barcode identifier sequence, wherein the standard histone or histone fragment and the standard polynucleotide form a stable protein-DNA association;
d) adding an affinity reagent to the doped library to capture an amount of native nucleosomes and standard comprising the epitope;
e) determining a relative genomic abundance for the epitope by comparing the amount of a given nucleotide sequence associated with the captured native nucleosomes comprising the epitope and the amount of a given nucleotide sequence associated with the native nucleosome in an input amount from the doped library;
f) determining a standard capture efficiency for the epitope by comparing the amount of a barcode identifier sequence associated with the captured standard and the amount of a given nucleotide sequence associated with the standard in an input amount from the doped library; and
g) determining the density of the epitope of the core histone at the genomic locus by comparing the relative genomic abundance to the standard capture efficiency;
thereby determining and quantitating the epigenetic or mutation status of the genomic locus.

3-8. (canceled)

9. The method of claim 1, wherein the biological sample comprises cells and the chromatin is isolated from the cells.

10. The method of claim 9, wherein the cells are cells from a tissue or organ affected by a disease or disorder associated with changes in histone post-translational modifications or DNA modifications.

11. The method of claim 9, wherein the cells are cells from a tissue or organ affected by a disease or disorder associated with mutations in histones.

12. The method of claim 1, wherein the biological sample is a biopsy.

13. The method of claim 9, wherein the cells are not cells from a tissue or organ affected by a disease or disorder associated with changes in histone post-translational modifications or DNA modifications.

14. The method of claim 9, wherein the cells are not cells from a tissue or organ affected by a disease or disorder associated with mutations in histones.

15. The method of claim 1, wherein the biological sample comprises peripheral blood mononuclear cells.

16. The method of claim 1, wherein the biological sample comprises circulating nucleosomes.

17. The method of claim 16, wherein the circulating nucleosomes are from blood cells.

18. The method of claim 16, wherein the circulating nucleosomes are from cells from a tissue or organ affected by a disease or disorder associated with changes in histone post-translational modifications or DNA modifications.

19. The method of claim 16, wherein the circulating nucleosomes are from cells from a tissue or organ affected by a disease or disorder associated with mutations in histones.

20. The method of claim 16, wherein the biological sample is plasma, urine, saliva, stool, lymphatic fluid, or cerebrospinal fluid.

21. The method of claim 1, wherein the subject is a human.

22. The method of claim 1, wherein determining the standard capture efficiency comprises comparing the ratio of a captured amount of the barcode identifier sequence to an input amount of the reconstituted nucleosomes.

23. The method of claim 1, wherein determining the relative genomic abundance comprises comparing the ratio of a captured amount of the native nucleosome nucleotide sequence to an input amount of native nucleosome nucleotide sequence.

24. The method of claim 1, wherein the affinity agent is an antibody directed towards the epitope.

25. The method of claim 1, wherein a plurality of standards is added to the library, each standard comprising a reconstituted nucleosome comprising (i) the standard histone or histone fragment having the epitope and (ii) the standard polynucleotide comprising the nucleosome positioning sequence and the barcode identifier sequence, wherein the barcode identifier sequence encodes a concentration parameter indicative of the concentration of the standard added to the library and wherein standards having equivalent concentrations are added to the library.

26. The method of claim 1, wherein a plurality of standards is added to the library, each standard comprising a reconstituted nucleosome comprising (i) the standard histone or histone fragment having the epitope and (ii) the standard polynucleotide comprising the nucleosome positioning sequence and the barcode identifier sequence, wherein the barcode identifier sequence encodes a concentration parameter indicative of the concentration of the standard added to the library and wherein standards having at least two differing concentrations are added to the library.

27. The method of claim 26, wherein standards having at least six differing concentrations are added to the library.

28. The method of claim 25, wherein the plurality of standards further comprises standards comprising reconstituted nucleosomes comprising (i) one or more off-target epitopes and (ii) a standard molecule barcode encoding an off-target epitope identity and concentration parameters indicative to the off-target epitope.

29. The method of claim 25, further comprising determining a specificity of off-target capture for the affinity reagent based on one or more capture efficiencies for the off-target epitopes and correcting the density of the epitope of the core histone at the genomic locus based on the specificity of off-target capture.

30. The method of claim 1, wherein the epitope is a post-translational modification or a protein isoform.

31. The method of claim 1, wherein the barcode identifier sequence is a sequence absent in the genome of the cell.

32. The method of claim 1, wherein an abundance of at least one of the polynucleotide comprising a nucleotide sequence indicative of the genomic locus and the standard polynucleotide is determined by a method selected from the group consisting of PCR, qPCR, ddPCR, next-generation sequencing, hybridization, autoradiography, fluorescent labeling, optical density and the use of intercalating fluorescent probes.

33. The method of claim 1, wherein the epitope of the core histone comprises at least one post-translational amino acid modification selected from the group consisting of N-acetylation of serine and alanine; phosphorylation of serine, threonine and tyrosine; N-crotonylation, N-acylation of lysine; N6-methylation, N6,N6-dimethylation, N6,N6,N6-trimethylation of lysine; omega-N-methylation, symmetrical-dimethylation, asymmetrical-dimethylation of arginine; citrullination of arginine; ubiquitinylation of lysine; sumoylation of lysine; O-methylation of serine and threonine, and ADP-ribosylation of arginine, aspartic acid and glutamic acid.

34. The method of claim 1, wherein the epitope of the core histone comprises at least one oncogenic mutation selected from the group consisting of H3K4M, H3K9M, H3K27M, and H3K36M.

35. The method of claim 1, wherein the standard polynucleotide is a double stranded polynucleotide.

36. The method of claim 35, wherein the double-stranded polynucleotide comprises a nucleotide sequence selected from the group consisting of a SEQ ID NOS: 1-115.

37. The method of claim 1, wherein the barcode identifier sequence comprises a molecule selected from the group consisting of a nucleotide barcode sequence molecule, a locked nucleic acid sequence and a DNA sequence.

38. The method of claim 1, wherein the disease or disorder associated with epigenetic modifications or mutations is a cancer, a central nervous system disorder, an autoimmune disorder, an inflammatory disorder, or an infection.

39. The method of claim 1, wherein the library of native nucleosomes comprises nucleosomes, each comprising the core histone and a polynucleotide comprising a nucleotide sequence indicative of its genomic locus of origin, and the method further comprises:
determining an amount of the core histone at the genomic locus in the doped library; and
determining an amount of standard in the doped library.

40. The method of claim 39, wherein determining the amount of the core histone at the genomic locus in the doped library comprises:
adding a second affinity reagent to the doped library to recover an amount of nucleosomes comprising a second epitope, wherein the second epitope is an invariant epitope present on the core histone, and
determining an amount of polynucleotide in the amount of recovered nucleosomes comprising the second epitope.

41. The method of claim 39, wherein determining the amount of standard in the doped library comprises:
recovering an amount of reconstituted nucleosome; wherein the reconstituted nucleosome comprises the second epitope, and
determining an amount of the standard molecule in the amount of recovered reconstituted nucleosomes comprising the second epitope.

42. The method of claim 41, wherein the affinity reagent is an antibody directed to the epitope and wherein the second affinity reagent is an antibody directed to the second epitope.

43-50. (canceled)

51. A kit comprising a panel of nucleosome standards, the panel comprising two or more nucleosomes comprising an epigenetic modification or a disease-associated histone mutation.

52-55. (canceled)


SEQUENCE LISTING (115 sequences)

SEQ ID NO: 1 - DNA, 203 bp, Artificial
  standard polynucleotide
ggcggccgacgcgatacaccgttcgtcgctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtgcgttcgacggtacgtcgagcggccgcc

SEQ ID NO: 2 - DNA, 203 bp, Artificial
  standard polynucleotide
ggcggcgtatcgcgtcgcgcgtaatcgactggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtgcgcgacgttacgctcgacgtagccgcc

SEQ ID NO: 3 - DNA, 203 bp, Artificial
  standard polynucleotide
ggcggcaccgatacgcgcgcggtacgatctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgttaatcgacgcgatatcgcgcgtgccgcc

SEQ ID NO: 4 - DNA, 203 bp, Artificial
  standard polynucleotide
ggcggcatatcgcgcgtcgtatcgcggtctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgttcgtatcgcgccgcgtattcgggccgcc

SEQ ID NO: 5 - DNA, 203 bp, Artificial
  standard polynucleotide
ggcggcccgcgcgatattacgcgcgaatctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtacgaacgtcgatcgtcgattcggccgcc

SEQ ID NO: 6 - DNA, 203 bp, Artificial
  standard polynucleotide
ggcggccgacgaacggttcgtacgcgagctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgttcgcgtacgaatcgcgtaatcggccgcc

SEQ ID NO: 7 - DNA, 203 bp, Artificial
  standard polynucleotide
ggcggccgcgtaatacgccgcgatacgactggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtaacgcgtatcgcgcgtaacgcggccgcc

SEQ ID NO: 8 - DNA, 203 bp, Artificial
  standard polynucleotide
ggcggccgtacgacgctcgcgatatccgctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtcgacgttaacgcgttacgcgtcgccgcc

SEQ ID NO: 9 - DNA, 203 bp, Artificial
  standard polynucleotide
ggcggcgcgttcgacgggtcgcgaactactggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtgtcgcgaactacgtcgttcgacgccgcc

SEQ ID NO: 10 - DNA, 203 bp, Artificial
  standard polynucleotide
ggcggctacgctcggactcgcgcgatgactggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtcgatcgtcgcatcggtacgctagccgcc

SEQ ID NO: 11 - DNA, 203 bp, Artificial
  standard polynucleotide
ggcggctattatgcgcgacccgcgtacgctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtcgtaccgcgatccgacgatcgagccgcc

SEQ ID NO: 12 - DNA, 203 bp, Artificial
  standard polynucleotide
ggcggctcgcgaccgtacgaatttcgcgctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtcgcgtcaatcgcgattacgcgagccgcc

SEQ ID NO: 13 - DNA, 203 bp, Artificial
  standard polynucleotide
ggcggctcgtacgaccgcgcgtatcgggctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtgcgatcgtacgcgcgacgttaagccgcc

SEQ ID NO: 14 - DNA, 203 bp, Artificial
  standard polynucleotide
ggcggcccgcgcgatattacgcgcgaatctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtcgacgttaacgcgttacgcgtcgccgcc

SEQ ID NO: 15 - DNA, 203 bp, Artificial
  standard polynucleotide
ggcggccgtacgacgctcgcgatatccgctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtacgaacgtcgatcgtcgattcggccgcc

SEQ ID NO: 16 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctattatgcgcgcgatacgcgtttc

SEQ ID NO: 17 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgcataataatcgcgcgatttc

SEQ ID NO: 18 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcatatcgcgcgttcgacgttcgttc

SEQ ID NO: 19 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcacgcgcgatattatcgcgtcgttc

SEQ ID NO: 20 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgtcgacgatcgtcgaatcgttc

SEQ ID NO: 21 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgtcgattcgacgcgaatcgttc

SEQ ID NO: 22 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctacgcgattcgtcgtttcgcgttc

SEQ ID NO: 23 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctatacgcgtcgacgattcgcgttc

SEQ ID NO: 24 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgcgtaatcgtttcgacgcgttc

SEQ ID NO: 25 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcttaacgtcgcgcgttcgaacgttc

SEQ ID NO: 26 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtattacgcgaatcgcgcgattc

SEQ ID NO: 27 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgattacgcgtcgcgcgtaattc

SEQ ID NO: 28 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtttcgtacgcgcgacgtaattc

SEQ ID NO: 29 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgcgtatacgtacgcgcgaattc

SEQ ID NO: 30 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgtaatacgcgcgaaattcgtc

SEQ ID NO: 31 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcgatagtcgacgttatcgcgtcgtc

SEQ ID NO: 32 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtacgaaacgcgttaacgtcgtc

SEQ ID NO: 33 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtcgactatctcgtcgtatcgtc

SEQ ID NO: 34 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcttacgcgtaccaacgcgtatcgtc

SEQ ID NO: 35 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgaatcgcgtattacgcgatcgtc

SEQ ID NO: 36 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcggtacgctatcgtacgatcgtc

SEQ ID NO: 37 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgacgcgtatacgaatttcgcgtc

SEQ ID NO: 38 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgacgcgataattacgtcgcgtc

SEQ ID NO: 39 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgcgcgaatattcgtatcgcgtc

SEQ ID NO: 40 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctatcgcgtcgagtgatatcgcgtc

SEQ ID NO: 41 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgtaatcgatacgttacgcgtc

SEQ ID NO: 42 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcttacgtcgcgataatcgacgcgtc

SEQ ID NO: 43 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctattcgcgcgatcgcgattacgtc

SEQ ID NO: 44 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgattacgcgaacgattcgacgtc

SEQ ID NO: 45 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtatacgcgattaacgcgacgtc

SEQ ID NO: 46 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctagcgtaccgacgacgttaacgtc

SEQ ID NO: 47 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcatcgtcgacgaacgttcgaacgtc

SEQ ID NO: 48 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgaatcgacgatagttcgcgactc

SEQ ID NO: 49 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgacgttaacgcgatatcactc

SEQ ID NO: 50 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcggtacgcgtaacgcgtcgattatc

SEQ ID NO: 51 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgcgacgtaaattcgcgcgtatc

SEQ ID NO: 52 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgtatcggtcgcgtaacgtatc

SEQ ID NO: 53 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgacgaacggtgtcgcgaactatc

SEQ ID NO: 54 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgaacggtcgtttcgcgcgatatc

SEQ ID NO: 55 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcccgacgatcgtacgacgcgatatc

SEQ ID NO: 56 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgacgtaccgtttacgcgtcgatc

SEQ ID NO: 57 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtacgacgctacgaacgtcgatc

SEQ ID NO: 58 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcccgcgcgatattttcgtcgcgatc

SEQ ID NO: 59 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgcgcgacatcgtaatcgcgatc

SEQ ID NO: 60 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgcgatatgattacgcgcgatc

SEQ ID NO: 61 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgtattcggttcgtacgcgatc

SEQ ID NO: 62 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgatcgtcggcgatcgtacgatc

SEQ ID NO: 63 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcacgatcgtcggtcgttcgacgatc

SEQ ID NO: 64 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgacgtatcggcgatacgacgatc

SEQ ID NO: 65 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcatatcgcgcggtcgtcgaacgatc

SEQ ID NO: 66 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgacgtaacggacgcgaaacgatc

SEQ ID NO: 67 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcacgaccgttcgcgtcgcgttaatc

SEQ ID NO: 68 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtatcggtcgcgatcgcgtaatc

SEQ ID NO: 69 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcctcgttcgtcgttcgcgcgtaatc

SEQ ID NO: 70 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcaccgttcgtcgtcgacgcgtaatc

SEQ ID NO: 71 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctacgtccgtcgcgacgcgataatc

SEQ ID NO: 72 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcccgttacgtcgtatcgcgcgaatc

SEQ ID NO: 73 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcacggtacgtcgttacgcgcgaatc

SEQ ID NO: 74 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcccgatacgtcgtcgcgtacgaatc

SEQ ID NO: 75 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgcacgatcgcgcgatacgaatc

SEQ ID NO: 76 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgccgaatcgacgcgtcgaaatc

SEQ ID NO: 77 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctatgcgtcgcgtcgcgacgaaatc

SEQ ID NO: 78 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccatatcgcgcgcgtatcgcggttc

SEQ ID NO: 79 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtatagcgcgccgtacgtcgttc

SEQ ID NO: 80 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcaccgatacgcgtagcgacgcgttc

SEQ ID NO: 81 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcccgaatacgcgtcgacgaccgttc

SEQ ID NO: 82 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgtacgaccgcggtcgaacgttc

SEQ ID NO: 83 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcagcgtcgtacgtcgcgacgagttc

SEQ ID NO: 84 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgctatacgcgtaccgcgattc

SEQ ID NO: 85 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgaccgatacgcgcggtacgattc

SEQ ID NO: 86 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcttcgagcgacgcggcgtacgattc

SEQ ID NO: 87 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcgtcgaacgacgcggtcgacgattc

SEQ ID NO: 88 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcgacgcgtaacgccgcgcgtaattc

SEQ ID NO: 89 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgacgcgtagcgcgacgcaattc

SEQ ID NO: 90 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgacgaacgagtcgtatcgcggtc

SEQ ID NO: 91 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgttacgcgtcttatcgcgcggtc

SEQ ID NO: 92 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctaacgtcgcgcattacgcgcggtc

SEQ ID NO: 93 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctacgctcggactatacgcgcggtc

SEQ ID NO: 94 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtcgttcgacacgacgtacggtc

SEQ ID NO: 95 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcgcgcgacgttacgattcgacggtc

SEQ ID NO: 96 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctgtcgcgcgtatacgctcgtcgtc

SEQ ID NO: 97 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcgtccgagcgtagtatcgcgtcgtc

SEQ ID NO: 98 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcgcgaccgtagttacgcgtcgtc

SEQ ID NO: 99 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgacggacgtacgtatccgtcgtc

SEQ ID NO: 100 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgacgcatagcgttacgtcgtc

SEQ ID NO: 101 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcctacgcgtcgacgcgttagtcgtc

SEQ ID NO: 102 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcccgacgatcgatcggcgtatcgtc

SEQ ID NO: 103 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgatcgtgcgacgcgactatcgtc

SEQ ID NO: 104 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgattcggcgatgcgacgatcgtc

SEQ ID NO: 105 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctacggtcgcgaccgtcgaatcgtc

SEQ ID NO: 106 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcatgtcgcgcgacgcgtcaatcgtc

SEQ ID NO: 107 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccggtcgtacgacgcgatatgcgtc

SEQ ID NO: 108 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctacgcgcgacacgtaatcggcgtc

SEQ ID NO: 109 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgtcgctcgaatatcggtcgcgtc

SEQ ID NO: 110 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcgcgttcgacggattgcgtcgcgtc

SEQ ID NO: 111 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgttacgcgcgatagtcgcgtc

SEQ ID NO: 112 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggccgcgtaacgcggtcgtatcgcgtc

SEQ ID NO: 113 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggctcggtacgcgccggatatcgcgtc

SEQ ID NO: 114 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcccgtcgaacgccgcatatcgcgtc

SEQ ID NO: 115 - DNA, 147 bp, Artificial
  standard polynucleotide
ctggagaatcccggtgccgaggccgctcaattggtcgtagacagctctagcaccgcttaaacgcacgtacgcgctgtcccccgcgttttaaccgccaaggggattactccctagtctccaggcgcgcgtaccgataccgatcgcgtc